(19) United States
(12) Patent Application Publication
(10) Pub. No.: US 2013/0085186 A1
(43) Pub. Date: Apr. 4, 2013

(54) COSMETIC PREPARATIONS BASED ON MOLECULARLY IMPRINTED POLYMERS

(71) Applicants: Volker Wendel, Seeheim-Jugenheim (DE); Oliver Brüggemann, Frankfurt Am Main (DE); Arne Ptock, Neuss (DE)

(72) Inventors: Volker Wendel, Seeheim-Jugenheim (DE); Oliver Brüggemann, Frankfurt Am Main (DE); Arne Ptock, Neuss (DE)

(73) Assignee: BASF SE, Ludwigshafen (DE)

(21) Appl. No.: 13/567,565

(22) Filed: Aug. 6, 2012

Related U.S. Application Data
(62) Division of application No. 12/441,757, filed on Mar. 18, 2009, now abandoned.

(30) Foreign Application Priority Data
Sep. 19, 2006 (EP) 06120922.7

(51) Int. Cl. A61K 8/81; A61Q 19/00; A61Q 5/06; A61Q 19/04; A61Q 5/02; A61Q 5/12; A61Q 17/04; A61Q 19/10; A61Q 1/00

(52) U.S. Cl. CPC A61K 8/8194; A61K 8/8164; A61K 8/817; A61Q 19/10; A61Q 19/00; A61Q 5/06; A61Q 1/00; A61Q 5/02; A61Q 5/12; A61Q 17/04; A61Q 19/04
USPC 514/772.5; 514/772.4; 514/772.6

(57) ABSTRACT

The present invention relates to cosmetic or dermatological formulations comprising at least one active compound, at least one polymer which is molecularly imprinted in the presence of this active compound and at least one fatty phase.

### CROSS REFERENCE TO RELATED APPLICATIONS

This application is a divisional application of U.S. application Ser. No. 12/441,757, filed Mar. 18, 2009, which is a National Stage of PCT/EP2007/059683 filed Sep. 14, 2007 which in turn claims priority from European Application 06120922.7 filed Sep. 19, 2006, the entire contents of all of which are incorporated herein by reference.

### DESCRIPTION

The present invention relates to cosmetic or dermatological formulations comprising at least one active compound, at least one polymer which is molecularly imprinted in the presence of this active compound and at least one fatty phase.

If particular actions of cosmetic products are to be achieved, the constituents are of decisive importance. The high level of available constituents and raw materials in cosmetic formulations is continuously being expanded, since consumers are interested in exacting and active products which, for example, can counteract the effects of ageing. The cosmetics manufacturers are also interested here in active compounds which are capable of revitalizing the skin or protecting it from the consequences of ageing due to the influences of light. Whereas in the past such substances primarily served to smooth and moisturize the skin, they are nowadays supplemented by a large number of various materials having a physiological action. Examples of these are vitamins, fruit acids or ceramides. The method and manner of stabilization of such active compounds is also of increasing importance here. In cosmetics, there is a great interest in active compounds which can be stored in a stable manner in aqueous or also in water-containing systems.

To maintain the activity of thermolabile, oxidation-sensitive or readily volatile cosmetic or dermatological active compounds, it is desirable to protect these within the formulation.

Release of cosmetic or dermatological active compounds which persists over a relatively long period of time, so-called controlled release, is often desirable.

Polymeric microencapsulations, which moreover can protect the active compounds, have proved useful, for example, for controlled release of cosmetic active compounds for some uses.

Ideally, however, the active compounds should be released not during storage of the formulation, but only at the proposed site of action.

The release of the active compounds from such microcapsules often takes place during use of the formulations containing them by destruction of the shell as a result of mechanical, thermal, chemical or enzymatic action. These opening variants usually are not without effect on the desired biological activity of the encapsulated active compounds.

In cosmetic formulations for treatment of skin, in particular sensitive or irritated skin and very especially in baby care, for obvious reasons it is problematic or impossible, however, to use such release mechanisms for the active compounds.

In skin care, it must furthermore be ensured that the acid protective covering of the skin is not damaged by unsuitable additives, but is maintained and assisted, i.e. the “natural” ambient conditions are largely maintained.

### BRIEF SUMMARY OF THE INVENTION

The object of the present invention was to provide formulations having a selective affinity for a particular active compound, from which this active compound is released gradually and gently only at the site of action.

It was furthermore an object of the present invention to provide formulations from which the release of an active compound is stimulated by physiological influences, such as, for example, by bringing into contact with skin or other organs. In other words, an object of the present invention was to render possible a controlled release of an active compound of a formulation, wherein the buffer capacity of the skin or of the body is sufficient as selective noxae for release of the active compound after use of the formulation.

The abovementioned objects are achieved by providing cosmetic or dermatological formulations comprising at least one active compound, at least one polymer which is molecularly imprinted in the presence of this active compound and at least one fatty phase.

### BRIEF DESCRIPTION OF THE DRAWINGS

FIG. 1 shows controlled release of DL-alpha-tocopherol from loaded trimethylolpropane trimethacrylate MIP particles-continuous extraction of the DL-alpha-tocopherol from the polymers with water of various pH values in ultrafiltration cells-plot of the absolute cumulative weights of DL-alpha-tocopherol released against time.

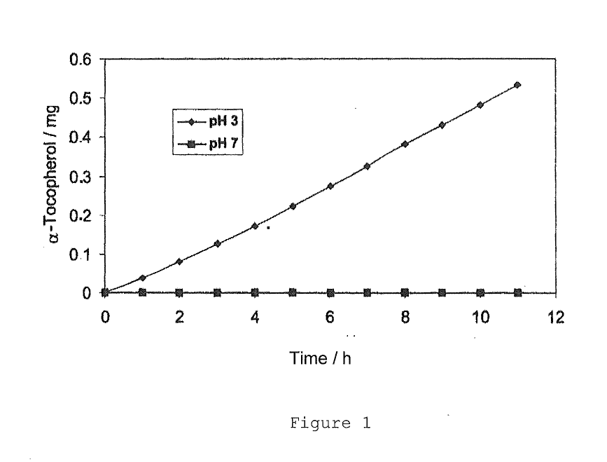

FIG. 2 shows controlled release of DL-alpha-tocopherol from loaded methacrylic acid/trimethylolpropane trimethacrylate MIP particles-extraction of the DL-alphat-tocopherol from the polymers with water of various pH values in batch reactors-plot of the DL-alpha tocopherol concentrations in the batch reactor against time.

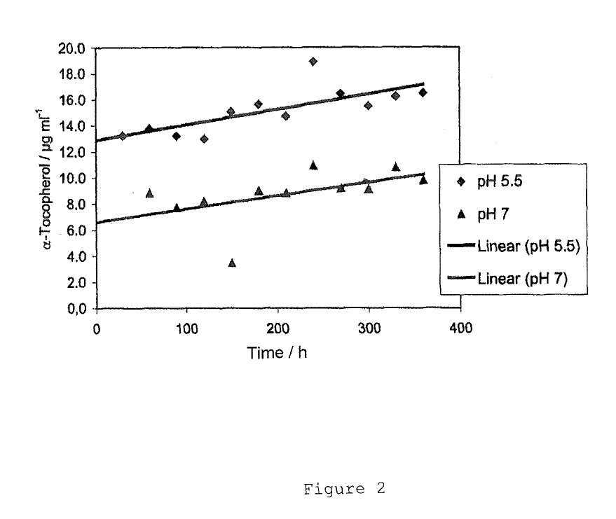

FIG. 3 shows controlled release of trichloroisocyanuric acid from methacrylic acid/trimethylolpropane trimethacrylate MIP particles-continuous extraction of the trichloroisocyanuric acid from the polymers with water of various pH values in ultrafiltration cells-plot of the absolute cumulative weights of trichloroisocyanuric acid released against time.

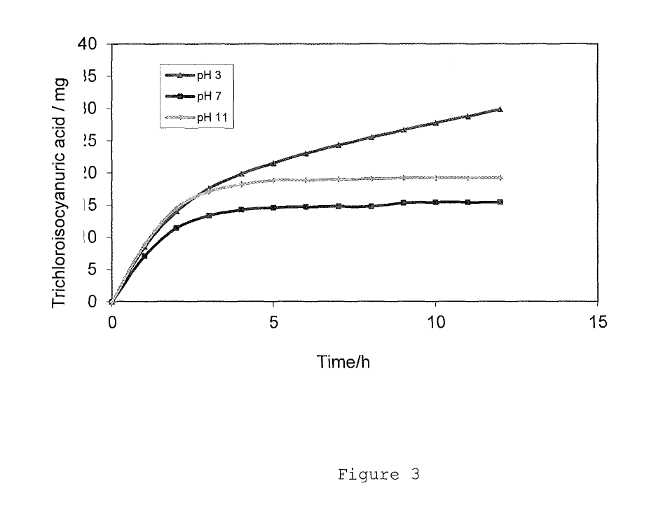

### DETAILED DESCRIPTION OF THE INVENTION

Formulations according to the invention render possible a controlled release of hydrophilic, amphiphilic, lipophilic, oxidation-sensitive or hydrolysis-sensitive active compounds. This applies to all conventional cosmetic and dermatological forms of application and presentation.

Where cosmetic active compounds, cosmetic formulations, cosmetic acceptance or cosmetic uses are described explicitly in the context of this invention and the corresponding dermatological forms are not also expressly mentioned, dermatological active compounds, formulations, acceptance and uses are likewise included in all cases.

Molecular imprinting is a technology which has been worked on intensively for some years, above all in the fields of chromatography, solid phase extraction and waste water treatment.

As literature which provides an overview there may be mentioned at this point “Molecular Imprinting—From Fundamentals to Applications”, Komiyama et al., Wiley-VCH, ISBN 3-527-30569-6 and ACS Symposium Series 703, “Molecular and Ionic Recognition with imprinted polymers”, ed. R. A. Bartsch and M. Maeda, ISBN 0-8412-3574-0.

Molecular imprinting of polymers is also known, for example, from the field of analysis of agricultural active compounds, e.g. J. Agric. Food Chem. 1995, 43, 1424-1427, Journal of Physics: Conference Series 10 (2005) 281-284, J. Agric. Food Chem. 1996, 44, 141-145, Chemistry Letters 7 (1995), 491-612.

Molecularly imprinted polymers are widely employed as stationary phases in high pressure liquid chromatography (HPLC) (see, for example, Molecular and Ionic Recognition with imprinted polymers, ACS Symposium Series 703, p. 5)

Kanekiyo et al. (Angew. Chem. Int. Ed. 2003, 42, 3014-16) describe molecularly imprinted polymers (MIPs), of which the affinity for the host molecules with which imprinting has been carried out is pH-dependent. A polymer based on acryloyl-amylose and monomers containing carboxyl groups and crosslinked with N,N-methylenebisacrylamide served as the MIP. Bisphenol A was used as the host molecule. It was demonstrated that for these MIPs containing COOH groups, the binding capacity for bisphenol A decreases with increasing pH.

Demirel et al. (Macromol. Biosci. 2005, 5, 1032-37) describe the pH- and temperature-dependency of the adsorption of bovine serum albumin (BSA) by hydrogels from N-tert-butylacrylamide/acrylamide/maleic acid copolymers.

Byrne et al. (Advanced Drug Delivery Reviews 54 (2002) 149-161) describe the use of molecularly imprinted hydrogels from controlled release gels for medicaments.

Cunliffe et al. (Advanced Drug Delivery Reviews 57 (2005) 1836-1853) likewise describe the use of molecularly imprinted hydrogels for the release of pharmaceutical active compounds.

EP-A 925776 describes molecularly imprinted polymers having bonding sites for at least one organoleptic substance. Cosmetic formulations which furthermore contain a fatty phase are not described.

The expression “at least one active compound” means that one, two or more cosmetic active compounds mentioned below can be used for the preparation of the molecularly imprinted polymer, preferably one or two, particularly preferably one.

The molecularly imprinted polymers comprise, in copolymerized form,

a) at least one compound having a double bond which can be polymerized by means of free-radical polymerization and
b) at least one compound having at least two non-conjugated double bonds which can be polymerized by means of free-radical polymerization.

In this context, compound a) is preferably chosen from

a1) anionic or anionogenic compounds which can be polymerized by means of free-radical polymerization
a2) esters of α,β-ethylenically unsaturated carboxylic acids
a3) amides of α,β-ethylenically unsaturated carboxylic acids
a4) esters of vinyl alcohol or allyl alcohol with C₁-C₃₀-monocarboxylic acids, vinyl ethers, vinyllactams, vinylimidazoles, vinylaromatics, vinyl halides, vinylidene halides, vinylpyridines, C₂-C₈-monoolefins, non-aromatic hydrocarbons having at least 2 conjugated double bonds and
a5) mixtures thereof.
Compounds a1)

The anionic or anionogenic compounds a1) which can be polymerized by means of free-radical polymerization include monoethylenically unsaturated mono- and dicarboxylic acids having 3 to 25, preferably 3 to 6 C atoms, which can also be employed in the form of their salts or anhydrides. Examples of these are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The compounds a1) furthermore include the half-esters of monoethylenically unsaturated dicarboxylic acids having 4 to 10, preferably 4 to 6 C atoms, e.g. maleic acid, such as maleic acid monomethyl ester.

Compounds a1) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosphonic acid.

The compounds a1) also include the salts of the abovementioned acids, in particular the sodium, potassium and ammonium salts, and the salts with cosmetically acceptable amines. Compounds a1) can be employed as such or as mixtures with one another.

Compound a1) is preferably chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof, particularly preferably acrylic acid, methacrylic acid and mixtures thereof, and in particular methacrylic acid.

Compounds a2)

Compound a2) is chosen, for example, from the group consisting of methyl (meth)acrylate, ethyl (meth)acrylate, n-propyl (meth)acrylate, i-propyl (meth)acrylate, n-butyl (meth)acrylate, tert-butyl (meth)acrylate, i-butyl (meth)acrylate, sec-butyl (meth)acrylate, 2-pentyl (meth)acrylate, 3-pentyl (meth)acrylate, isopentyl (meth)acrylate, neopentyl (meth)acrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arachidyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate, phenoxyethyl acrylate, 4-t-butylcyclohexyl acrylate, cyclohexyl (meth)acrylate, ureido (meth)acrylate, tetrahydrofurfuryl (meth)acrylate and mixtures thereof.

Compound a2) is preferably chosen from the esters of (meth)acrylic acid.

Compound a2) is particularly preferably chosen from methacrylates and acrylates. Preferred (meth)acrylates are C₁-C₁₀-alkyl (meth)acrylates, and in particular the abovementioned C₁-C₄-alkyl (meth)acrylates.

Suitable compounds a2) are also the esters of α,β-ethylenically unsaturated mono- and dicarboxylic acids with amino alcohols. Preferred amino alcohols are C₂-C₁₂-amino alcohols which are C₁-C₈-mono- or -dialkylated on the amine nitrogen. Suitable acid components of these esters are e.g. acrylic acid, methacrylic acid, fumaric acid, maleic acid, itaconic acid, crotonic acid, maleic anhydride, monobutyl maleate and mixtures thereof. Acrylic acid, methacrylic acid and mixtures thereof are preferably employed as the acid component.

Preferred monomers a3) are N-tert-butylaminoethyl (meth)acrylate, N,N-dimethylaminomethyl (meth)acrylate, N,N-dimethylaminoethyl (meth)acrylate, N,N-diethylaminoethyl (meth)acrylate, N,N-dimethylaminopropyl (meth)acrylate, N,N-diethylaminopropyl (meth)acrylate and N,N-dimethylaminocyclohexyl (meth)acrylate. N-tert-Butylaminoethyl (meth)acrylate and N,N-dimethylaminoethyl (meth)acrylate are particularly preferred.

Suitable compounds a3) are also esters of α,β-ethylenically unsaturated mono- and dicarboxylic acids with diols, such as, for example, 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate.

Compounds a3)

Compounds a3) can preferably be chosen from the group consisting of acrylamide, methacrylamide, N-methyl(meth)acrylamide, N-ethyl(meth)acrylamide, N-propyl(meth)acrylamide, N-(n-butyl)(meth)acrylamide, N-(tert-butyl)(meth)acrylamide, N,N-dimethyl(meth)acrylamide, N,N-diethyl(meth)acrylamide, piperidinyl(meth)acrylamide and morpholinyl(meth)acrylamide, N-(n-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arachidyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide and N-lauryl(meth)acrylamide.

Suitable monomers a3) are also the amines of the abovementioned α,β-ethylenically unsaturated mono- and dicarboxylic acids with diamines which contain at least one primary or secondary amino group. Diamines which contain one tertiary and one primary or secondary amino group are preferred.

Suitable monomers a3) are e.g. N-tert-butylaminoethyl(meth)acrylamide, N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)-butyl]methacrylamide, N-[2-(diethylamino)ethyl]-acrylamide, N-[4-(dimethylamino)cyclohexyl]acrylamide and N-[4-(dimethylamino)cyclohexyl]methacrylamide.

Suitable monomers a3) are also the hydroxyalkylamides of the abovementioned α,β-ethylenically unsaturated mono- and dicarboxylic acids, such as, for example, 2-hydroxyethylacrylamide, 2-hydroxyethylmethacrylamide, 2-hydroxyethylethacrylamide, 2-hydroxypropylacrylamide, 2-hydroxypropylmethacrylamide, 3-hydroxypropylacrylamide, 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide.

Compounds a4)

Suitable compounds a4) are, for example, N-vinyllactams and derivatives thereof, which can contain e.g. one or more C₁-C₆-alkyl substituents, such as methyl, ethyl, n-propyl, isopropyl, n-butyl, sec-butyl, tert-butyl etc. These include e.g. N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam and N-vinyl-7-ethyl-2-caprolactam. N-Vinylpyrrolidone and/or N-vinylcaprolactam are particularly preferably employed.

Suitable compounds a4) are, for example, also N-vinylimidazole compounds of the general formula (II)

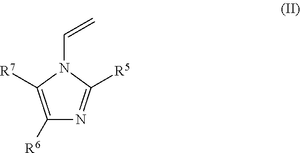

wherein R⁵ to R⁷ independently of one another represent hydrogen, C₁-C₄-alkyl or phenyl.

Examples of compounds of the general formula (II) are to be found in the following Table 1:

TABLE 1

| R⁵ | R⁶ | R⁷ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

1-Vinylimidazole (N-vinylimidazole) is preferred as monomer b).

### Compounds b)

Compounds b) are compounds having at least two non-conjugated double bonds which can be polymerized by means of free-radical polymerization. These compounds b) are also called, conventionally and in the following, crosslinking agents.

Suitable compounds b) are, for example, acrylates, methacrylates, allyl ethers or vinyl ethers of at least dihydric alcohols. In this context, the OH groups of the underlying alcohols can be completely or partly etherified or esterified; however, the crosslinking agents contain at least two ethylenically unsaturated groups.

Examples of the underlying alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentylglycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic acid neopentylglycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thio-pentane-1,5-diol and polyethylene glycols, polypropylene glycols and polytetrahydrofurans having molecular weights of in each case from 200 to 10,000. In addition to the homopolymers of ethylene oxide or propylene oxide, block copolymers of ethylene oxide or propylene oxide or copolymers which contain incorporated ethylene oxide and propylene oxide groups can also be employed. Examples of the underlying alcohols having more than two OH groups are trimethylolpropane, glycerol, pentaerythritol, 1,2,5-pentanetriol, 1,2,6-hexanetriol, triethoxycyanuric acid, sorbitan and sugars, such as sucrose, glucose and mannose. The polyhydric alcohols can of course also be employed as the corresponding ethoxylates or propoxylates, after reaction with ethylene oxide or propylene oxide. The polyhydric alcohols can also be first converted into the corresponding glycidyl ethers by reaction with epichlorohydrin. Ethylene glycol di(meth)acrylate and polyethylene glycol di(meth)acrylates are preferred.

Further suitable compounds b) are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, the monohydric, unsaturated alcohols can also be esterified with polybasic carboxylic acids, for example malonic acid, tartaric acid, trimellitic acid, phthalic acid, terephthalic acid, citric acid or succinic acid.

Further suitable compounds b) are esters of unsaturated carboxylic acids with the polyhydric alcohols described above, for example oleic acid, crotonic acid, cinnamic acid or 10-undecenoic acid.

Suitable compounds b) are moreover straight-chain or branched, linear or cyclic, aliphatic or aromatic hydrocarbons which have at least two double bonds, which should not be conjugated in the case of aliphatic hydrocarbons, e.g. divinylbenzene, divinyltoluene, 1,7-octadiene, 1,9-decadiene, 4-vinyl-1-cyclohexane, trivinylcyclohexane or polybutadienes having molecular weights of from 200 to 20,000.

Suitable compounds b) are furthermore the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. The amides of allylamine and unsaturated carboxylic acids, such as acrylic acid, methacrylic acid, itaconic acid or maleic acid, or at least dibasic carboxylic acids, such as have been described above, are likewise suitable.

Triallylamines and triallylmonoalkylammonium salts, e.g. triallylmethylammonium chloride or methyl-sulfate, are furthermore suitable as compounds b).

N-Vinyl compounds of urea derivatives, at least difunctional amides, cyanurates or urethanes, for example of urea, ethyleneurea, propyleneurea or tartaric acid diamide, e.g. N,N′-divinylethyleneurea or N,N′-divinylpropylene urea, are also suitable.

Further suitable compounds b) are divinyldioxane, tetraallylsilane or tetravinylsilane.

Mixtures of the abovementioned compounds b) can of course also be employed.

Ethylene glycol di(meth)acrylate, polyethylene glycol di(meth)acrylates, pentaerythritol triallyl ether, methylenebisacrylamide, N,N′-divinylethyleneurea, triallylamine, trimethylolpropane tri(meth)acrylate and triallylmonoalkylammonium salts are very particularly preferred as compounds b).

The molar ratio of compound a) to compound b) is as a rule in the range of from 1:2 to 1:10, preferably in the range of from 1:2 to 1:4, very particularly preferably in the range of from 1:2.5 to 1:3.5, and in particular in the range of from 1:2.8 to 1:3.2.

The novel polymers described here are prepared in the presence of the active compound (template) via a precipitation polymerization with a large excess of solvent (weight ratio of solvent to active compound in the range of from 100:1 to 5:1, preferably from 100:1 to 50:1, particularly preferably 59:1) in a batch or semi-batch process. The polymer particles obtained are purified by a Soxhlet extraction. After the active bonding sites (the molecular imprints) have been loaded again with the template (or if a template extraction has been omitted after the polymer synthesis), the polymer can be employed in order to release the cosmetic active compound in a controlled or retarded manner.

The formulations according to the invention are distinguished in that the rate of release of the active compound from the polymer-active compound complex is higher at pH 5 than at pH 7.

The polymer-active compound complex is obtained when the polymer has been imprinted with the active compound and non-bonded active compound has then been removed, or previously imprinted polymer which has been freed from bonded active compound is loaded with active compound again.

The rate of release is understood as meaning that amount of active compound which is released from the polymer-active compound complex per unit time; the rate can be stated, for example, in μg*min⁻¹.

The rate of release is determined as follows: an ultrafiltration cell is filled with a dispersion of 100 mg of the molecularly imprinted polymer in 100 ml of water (of adjusted pH) and the mixture is stirred for 15 minutes until homogenized. The extraction agent (likewise water of the same adjusted pH) is passed into the cell from the reservoir via the feed by means of a hose pump. The extract discharged from the cell is passed into a collecting vessel on the under-side of the cell. Experiment run time: 12 hours; changing of the collecting vessel: every 60 minutes; 12 fractions are thus collected over the experiment time. The volume, weight and removal period of the individual fractions are determined and a sample of 2 ml is taken from each fraction. These samples are filtered over a 0.45 μl filter, transferred into injection bottles and employed for the tocopherol determination by means of HPLC.

The present invention also relates to the process for the preparation of the molecularly imprinted polymers, which comprises preparing the polymer by precipitation polymerization in the presence of an active compound.

The fundamental principle of precipitation polymerization is familiar to the person skilled in the art and is described, for example, in Guyot, A. (1989), in: Comprehensive Polymer Science, vol. 4: Eastmond, G. C., Ledwith, A., Russo, S., Sigwalt, P. (eds.). Oxford: Pergamon, pp. 261-273.

In a preferred embodiment, the molecularly imprinted polymer can be prepared by a process in which
(a) at least one compound a) is mixed with at least one active compound in a suitable solvent, at least one compound b) is added and the polymerization is started, compound b) preferably having been dissolved beforehand in a solvent which, in a very particularly preferred embodiment, corresponds to the solvent in which compound a) is dissolved, or
(b) at least one compound a) is mixed with at least one active compound and at least one compound b) in a suitable solvent and the polymerization is then started.

The polymerization can take place in a free-radical, anionic, cationic or coordinative mechanism or in accordance with the principle of a polycondensation or polyaddition. The polymerization preferably takes place via a free-radical mechanism. In this context, various initiators and/or catalysts can be employed, optionally also in combination with a supply of heat.

For cationic polymerizations, the following initiators, for example, can be used:

proton acids, Lewis acids with and without coinitiators, carbonium ions, iodonium ions and/or ionizing radiation.

For anionic polymerizations, the following initiators can be used: bases, Lewis bases, organometallic compounds and/or electron transfer agents, e.g. alkali metals, alkali metal-aromatic complexes or metal ketyls.

For coordinative polymerizations, the following initiators/catalysts can be employed:

organometallic mixed catalysts (Ziegler-Natta catalysts), π complexes with transition metals, e.g. metallocenes, and/or activated transition metal oxides.

Suitable initiators for the preferred free-radical polymerization are, for example, peroxides or azo compounds, substituted ethanes (e.g. benzopinacols), redox systems with inorganic and organic components, heat, UV light and other high-energy radiation, hydroperoxides, peresters and persulfates, such as e.g. potassium peroxodisulfate, preferably azo compounds.

Suitable azo compounds are 2,2′-azobisisobutyronitrile, 2,2′-azobis(2-methylbutyronitrile), 2,2′-azobis(2,4-dimethylvaleronitrile), 2,2′-azobis(4-methoxy-2,4-dimethylvaleronitrile), 1,1′-azobis(1-cyclohexanecarbonitrile), 2,2′-azobis(isobutyramide)dihydrate, 2-phenylazo-2,4-dimethyl-4-methoxyvaleronitrile, dimethyl 2,2′-azobisisobutyrate, 2-(carbamoylazo)-isobutyronitrile, 2,2′-azobis(2,4,4-trimethylpentane), 2,2′-azobis(2-methylpropane), 2,2′-azobis(N,N′-dimethyleneisobutyramidine), as the free base or as the hydrochloride, 2,2′-azobis(2-amidinopropane), as the free base or as the hydrochloride, 2,2′-azobis(2-methyl-N-[1,1 bis(hydroxymethyl)ethyl]propionamide or 2,2′-azobis(2-methyl-N-[1,1-bis(hydroxymethyl)-2-hydroxyethyl]propionamide.

Suitable peroxides are, for example, acetylcyclohexanesulfonic acid peroxide, diisopropyl peroxydicarbonate t-amyl perneodecanoate, t-butyl perneodecanoate, t-butyl perpivalate, t-amyl perpivalate, bis(2,4-dichlorbenzoic acid) peroxide, di-isononanoic acid peroxide, di-decanoic acid peroxide, dioctanoic acid peroxide, dilauric acid peroxide, bis(2-methylbenzoic acid) peroxide, disuccinic acid peroxide, diacetyl peroxide, dibenzoic acid peroxide, t-butyl per-2-ethylhexanoate, bis-(4-chlorobenzoic acid) peroxide, t-butyl perisobutyrate, t-butyl permaleate, 1,1-bis(t-butylperoxy)-3,5,5-trimethylcyclohexane, 1,1-bis(t-butylperoxy)cyclohexane, t-butyl peroxyisopropyl-carbonate, t-butyl perisononanoate, t-butyl peracetate, t-amyl perbenzoate, t-butyl perbenzoate, 2,2-bis(t-butylperoxy)butane, 2,2 bis-10-(t-butylperoxy)propane, dicumyl peroxide, 2,5-dimethylhexane-2,5-di-t-butyl peroxide, 3-t-butyl peroxy-3-phenylphthalide, di-t-amyl peroxide, α,α′-bis(t-butylperoxyisopropyl)-benzene, 3,5-bis(t-butylperoxy)-3,5-dimethyl-1,2-dioxolane, di-t-butyl peroxide, 2,5-dimethylhexyne-2,5-di-t-butyl peroxide, 3,3,6,6,9,9-hexamethyl-1,2,4,5-tetraoxacyclononane, p-menthane hydroperoxide, pinane hydroperoxide, diisopropylbenzene monohydroperoxide, cumene hydroperoxide or t-butyl hydroperoxide.

Depending on the state of aggregation, the crosslinking agent can be added to the reaction mixture in the solid or liquid form, or added in dissolved or dispersed form (i.e. emulsified or suspended) in a solvent, preferably in dissolved form. Preferably, a liquid crosslinking agent or a crosslinking agent dissolved (or dispersed/mixed) in a solvent is added to the reaction mixture, particularly preferably a crosslinking agent dissolved (or dispersed/mixed) in a solvent. In a very particularly preferred embodiment, the crosslinking agent is dissolved in the same solvent as the functional monomer or the crosslinking agent.

Solvents which can be used are organic solvents, for example dimethylformamide, ethanol, methanol, isopropanol, chloroform, methylene chloride, toluene, dimethylsulfoxide, hexane and acetonitrile, preferably toluene and acetonitrile. Mixtures of the abovementioned solvents can also be used.

In a further embodiment, up to a proportion of 50 wt. % of water can be added to the solvent or the solvent mixtures.

As a rule, the polymerization is carried out at temperatures of 40-120° C., depending on the solvent.

All the embodiments of the abovementioned particles are called “MIP” in the following.

The particles obtained during the polymerization either can be employed directly in the formulations, or formulation auxiliaries can be added to them and they can be formulated accordingly.

The present invention also provides the use of molecularly imprinted polymers in cosmetic formulations, in particular skin cosmetic formulations.

The present invention also provides a method for treatment of keratin surfaces, which comprises bringing the keratin surface into contact with a molecularly imprinted polymer.

### Active Compounds

The formulations according to the invention comprise cosmetically acceptable active compounds. These active compounds are released in particular in the pH range of from 5 to 7 in a controlled manner from the combination with the polymer molecularly imprinted with this active compound.

According to the invention, the active compounds (one or more compounds) can advantageously be chosen from the group consisting of acetylsalicylic acid, atropine, azulene, hydrocortisone and derivatives thereof, e.g. hydrocortisone 17-valerate, vitamins of the B and D series, in particular vitamin B₁, vitamin B₁₂, vitamin D, vitamin A and derivatives thereof, such as retinyl palmitate, vitamin E or derivatives thereof, such as e.g. tocopheryl acetate, vitamin C and derivatives thereof, such as e.g. ascorbyl glucoside, and also niacinamide, panthenol, bisabolol polydocanol, unsaturated fatty acids, such as e.g. the essential fatty acids (conventionally called vitamin F), in particular γ-linolenic acid, oleic acid, eicosapentaenoic acid, docosahexaenoic acid and derivatives thereof, chloramphenicol, caffeine, prostaglandins, thymol, camphor, squalene, extracts or other products of plant and animal origin, e.g. evening primrose oil, borage oil or carob bean oil, fish oils, cod-liver oil, and also ceramides and ceramide-like compounds, incense extract, green tea extract, water lily extract, liquorice extract and witch hazel, antidandruff active compounds (e.g. selenium disulfide, zinc pyrithione, piroctone, olamine, climbazole, octopirox, polydocanol and combinations thereof), and complexed active compounds, such as e.g. those of γ-oryzanol and calcium salts, such as calcium pantothenate, calcium chloride and calcium acetate.

The active compound or compounds can also be chosen from the group consisting of NO synthase inhibitors, in particular if the compositions according to the invention are to be used for treatment and prophylaxis of the symptoms of intrinsic and/or extrinsic ageing of the skin and for treatment and prophylaxis of the harmful effects of ultraviolet radiation on the skin and hair. Nitroarginine is the preferred NO synthase inhibitor.

The active compound or compounds are furthermore advantageously chosen from the group including catechols and bile acid esters of catechols and aqueous or organic extracts from plants or plant parts which have a content of catechols or bile acid esters of catechols, such as, for example, the leaves of the Theaceae plant family, in particular of the species Camellia sinensis (green tea). Typical constituents thereof (e.g. polyphenols or catechols, caffeine, vitamins, sugars, minerals, amino acids, lipids) are particularly advantageous.

Catechols are a group of compounds which are to be interpreted as hydrogenated flavones or anthocyanidines and are derivatives of “catechol” (catechol, 3,3′,4′,5,7-flavanepentaol, 2-(3,4-dihydroxyphenyl)-chromane-3,5,7-triol). Epicatechol ((2R,3R)-3,3′,4′,5,7-flavanepentaol) is also an advantageous active compound in the context of the present invention.

Plant extracts having a content of catechols, in particular extracts of green tea, such as e.g. extracts from leaves of the plants of the species Camellia spec., very particularly the tea varieties Camellia sinensis, C. assamica, C. taliensis or C. inawadiensis, and cross-varieties of these with, for example, Camellia japonica, are furthermore advantageous.

Preferred active compounds are furthermore polyphenols or catechols from the group consisting of (−)-catechol, (+)-catechol, (−)-catechol gallate, (−)-gallocatechol gallate, (+)-epicatechol, (−)-epicatechol, (−)-epicatechol gallate, (−)-epigallocatechol and (−)-epigallocatechol gallate.

Flavone and its derivatives (often also collectively called “flavones”) are also advantageous active compounds in the context of the present invention. They are characterized by the following basic structure (substitution positions shown):

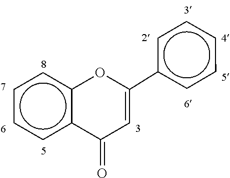

Some of the more important flavones, which can also preferably be employed in compositions according to the invention, are listed in the following Table 2.

TABLE 2
Table 2: Flavones

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones as a rule occur in the glycosidated form.

According to the invention, the flavonoids are preferably chosen from the group consisting of substances of the general formula

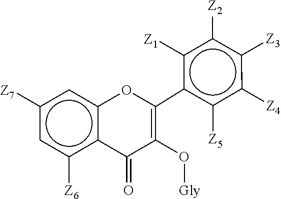

wherein Z₁ to Z₇ are chosen independently of one another from the group consisting of H, OH, alkoxy- and hydroxyalkoxy-, wherein the alkoxy and hydroxyalkoxy groups can be branched and unbranched and can contain 1 to 18 C atoms, and wherein Gly is chosen from the group consisting of mono- and oligoglycoside radicals.

According to the invention, however, the flavonoids can also advantageously be chosen from the group consisting of substances of the general formula

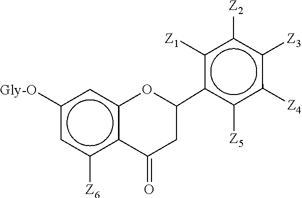

wherein Z₁ to Z₆ are chosen independently of one another from the group consisting of H, OH, alkoxy- and hydroxyalkoxy-, wherein the alkoxy and hydroxyalkoxy groups can be branched and unbranched and can contain 1 to 18 C atoms, and wherein Gly is chosen from the group consisting of mono- and oligoglycoside radicals.

Such structures can preferably be chosen from the group consisting of substances of the general formula

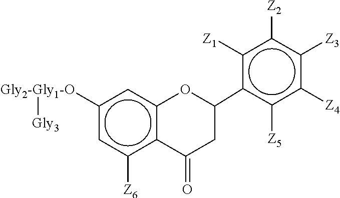

wherein Z₁ to Z₆ independently of one another are as mentioned above and Gly₂ and Gly₃ independently of one another are monoglycoside radicals or oligoglycoside radicals. Gly₂ and Gly₃ can also individually or together represent saturation by hydrogen atoms.

Preferably, Gly₁, Gly₂ and Gly₃ are chosen independently of one another from the group consisting of hexosyl radicals, in particular rhamnosyl radicals and glucosyl radicals. However, other hexosyl radicals, for example allosyl, altrosyl, galactosyl, gulosyl, idosyl, mannosyl and talosyl, are also optionally advantageously to be used.

It may also be advantageous according to the invention to use pentosyl radicals.

Advantageously, Z₁ to Z₅ are chosen independently of one another from the group consisting of H, OH, methoxy-, ethoxy- and 2-hydroxyethoxy-, and the flavone glycosides correspond to the general structural formula

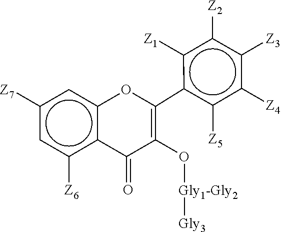

The flavone glycosides are particularly advantageously chosen from the group represented by the following structure,

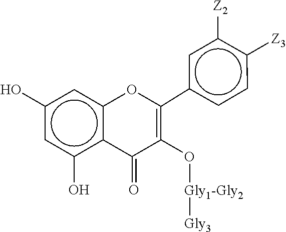

wherein Gly₁, Gly₂ and Gly₃ independently of one another represent monoglycoside radicals or oligoglycoside radicals. Gly₂ and Gly₃ can also individually or together represent saturation by hydrogen atoms.

Preferably, Gly₁, Gly₂ and Gly₃ are chosen independently of one another from the group consisting of hexosyl radicals, in particular rhamnosyl radicals and glucosyl radicals. However, other hexosyl radicals, for example allosyl, altrosyl, galactosyl, gulosyl, idosyl, mannosyl and talosyl, are also optionally advantageously to be used.

It may also be advantageous according to the invention to use pentosyl radicals.

It is particularly advantageous in the context of the present invention to choose the flavone glycoside or glycosides from the group consisting of α-glucosylrutin, α-glucosylmyricetin, α-glucosylisoquercitrin, α-glucosylisoquercetin and α-glucosylquercitrin.

Further advantageous active compounds are sericoside, pyridoxol, vitamin K, biotin and aroma substances.

The active compounds (one or more compounds) can furthermore also very advantageously be chosen from the group consisting of hydrophilic active compounds, in particular from the following group:

α-hydroxy acids, such as lactic acid or salicylic acid, or salts thereof, such as e.g. Na lactate, Ca lactate or TEA lactate, urea, allantoin, serine, sorbitol, glycerol, milk proteins, panthenol or chitosan.

The active compounds (one or more compounds) can furthermore also very advantageously be chosen from the group consisting of light filter active compounds.

Suitable light filter active compounds are substances which absorb UV rays in the UV-B and/or UV-A range. By these there are to be understood organic substances which are capable of absorbing ultraviolet rays and of releasing the energy absorbed again in the form of longer-wavelength radiation, e.g. heat. The organic substances can be oil-soluble or water-soluble. Suitable UV filters are e.g. 2,4,6-triaryl-1,3,5-triazines, in which the aryl groups in each case can carry at least one substituent, which is preferably chosen from hydroxyl, alkoxy, specifically methoxy, and alkoxycarbonyl, specifically methoxycarbonyl and ethoxycarbonyl. p-Aminobenzoic acid esters, cinnamic acid esters, benzophenones, camphor derivatives and pigments which keep out UV rays, such as titanium dioxide, talc and zinc oxide, are furthermore suitable. Pigments based on titanium dioxide are particularly preferred.

Oil-soluble UV-B filters which can be used are e.g. the following substances:

3-benzylidenecamphor and derivatives thereof, e.g. 3-(4-methylbenzylidene)camphor;
4-aminobenzoic acid derivatives, preferably 4-(dimethylamino)-benzoic acid 2-ethylhexyl ester, 4-(dimethylamino)benzoic acid 2-octyl ester and 4-(dimethylamino)benzoic acid amyl ester;
esters of cinnamic acid, preferably 4-methoxycinnamic acid 2-ethylhexyl ester, 4-methoxycinnamic acid propyl ester, 4-methoxycinnamic acid isoamyl ester, 4 methoxycinnamic acid isopentyl ester and 2-cyano-3-phenyl-cinnamic acid 2-ethylhexyl ester (octocrylene);
esters of salicylic acid, preferably salicylic acid 2-ethylhexyl ester, salicylic acid 4-isopropylbenzyl ester and salicylic acid homomethyl ester;
derivatives of benzophenone, preferably 2-hydroxy-4-methoxybenzophenone, 2-hydroxy-4-methoxy-4′-methylbenzophenone and 2,2′-dihydroxy-4-methoxybenzophenone;
esters of benzalmalonic acid, preferably 4-methoxybenzalmalonic acid di-2-ethylhexyl ester;
triazine derivatives, such as e.g. 2,4,6-trianilino-(p-carbo-2′-ethyl-1′-hexyloxy)-1,3,5-triazine (octyltriazone) and dioctyl butamido triazone (Uvasorb® HEB);
propane-1,3-diones, such as e.g. 1-(4-tert-butylphenyl)-3-(4′-methoxyphenyl)propane-1,3-dione.

Possible water-soluble substances are:

2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and its salts;
sulphonic acid derivatives of 3-benzylidenecamphor, such as e.g. 4-(2-oxo-3-bornylidenemethyl)benzenesulphonic acid and 2-methyl-5-(2-oxo-3-bornylidene)sulphonic acid and salts thereof.

The use of esters of cinnamic acid, preferably 4-methoxycinnamic acid 2-ethylhexyl ester, 4-methoxycinnamic acid isopentyl ester and 2-cyano-3-phenyl-cinnamic acid 2-ethylhexyl ester (octocrylene), is particularly preferred.

The use of derivatives of benzophenone, in particular 2-hydroxy-4-methoxybenzophenone, 2-hydroxy-4-methoxy-4′-methylbenzophenone and 2,2′-dihydroxy-4-methoxybenzophenone, and the use of propane-1,3-diones, such as e.g. 1-(4-tert-butylphenyl)-3-(4′-methoxyphenyl)propane-1,3-dione, is furthermore preferred.

Possible typical UV-A filters are:

derivatives of benzoylmethane, such as, for example, 1-(4′-tert-butylphenyl)-3-(4′-methoxyphenyl)-propane-1,3-dione, 4-tert-butyl-4′-methoxydibenzoylmethane or 1-phenyl-3-(4′-isopropylphenyl)-propane-1,3-dione;
amino-hydroxy-substituted derivatives of benzophenones, such as e.g. N,N-diethylamino-hydroxybenzoyl-n-hexyl-benzoate.

The UV-A and UV-B filters can of course also be employed in mixtures.

Further suitable UV filter substances are mentioned in the following Table 3.

TABLE 3
Suitable sunscreen agents

| | | CAS no. |
|---|---|---|
| No. | Substance | (= acid) |
| 1 | 4-aminobenzoic acid | 150-13-0 |
| 2 | 3-(4′-trimethylammonium)- | 52793-97-2 |
| | benzylidenebornan-2-one methyl-sulfate | |
| 3 | 3,3,5-trimethyl-cyclohexyl salicylate | 118-56-9 |
| | (homosalatum) | |
| 4 | 2-hydroxy-4-methoxy- | 131-57-7 |
| | benzophenone (oxybenzonum) | |
| 5 | 2-phenylbenzimidazole-5- | 27503-81-7 |
| | sulfonic acid and its potassium, | |
| | sodium and triethanolamine salts | |
| 6 | 3,3′-(1,4-phenylenedimethine)- | 90457-82-2 |
| | bis(7,7-dimethyl-2- | |
| | oxobicyclo[2.2.1]heptane-1- | |
| | methanesulfonic acid) and its salts | |
| 7 | 4-bis(polyethoxy)amino-benzoic | 113010-52-9 |
| | acid polyethoxy-ethyl ester | |
| 8 | 4-dimethylamino-benzoic acid 2-ethylhexyl ester | 21245-02-3 |
| 9 | salicylic acid 2-ethylhexyl ester | 118-60-5 |
| 10 | 4-methoxy-cinnamic acid 2-isoamyl ester | 71617-10-2 |
| 11 | 4-methoxy-cinnamic acid 2-ethylhexyl ester | 5466-77-3 |
| 12 | 2-hydroxy-4-methoxy-benzophenone-5-sulfonic acid | 4065-45-6 |
| | (sulisobenzonum) and the sodium salt | |
| 13 | 3-(4′-sulfobenzylidene)-bornan-2-one and salts | 58030-58-6 |
| 14 | 3-benzylidenebornan-2-one | 16087-24-8 |
| 15 | 1-(4′-isopropylphenyl)-3-phenylpropane-1,3-dione | 63260-25-9 |
| 16 | 4-isopropylbenzyl salicylate | 94134-93-7 |
| 17 | 3-imidazol-4-yl-acrylic acid and its ethyl ester | 104-98-3 |
| 18 | 2-cyano-3,3-diphenylacrylic acid ethyl ester | 5232-99-5 |
| 19 | 2-cyano-3,3-diphenylacrylic acid 2′-ethylhexyl ester | 6197-30-4 |
| 20 | menthyl-o-aminobenzoate or: | 134-09-8 |
| | 5-methyl-2-(1-methylethyl)-2-aminobenzoate | |
| 21 | glyceryl p-aminobenzoate or: | 136-44-7 |
| | 4-aminobenzoic acid 1-glyceryl ester | |
| 22 | 2,2′-dihydroxy-4- | 131-53-3 |
| | methoxybenzophenone (dioxybenzone) | |
| 23 | 2-hydroxy-4-methoxy-4-methylbenzophenone | 1641-17-4 |
| | (mexenone) | |
| 24 | triethanolamine salicylate | 2174-16-5 |
| 25 | dimethoxyphenylglyoxalic acid or: | 4732-70-1 |
| | sodium 3,4-dimethoxy-phenyl-glyoxalate | |
| 26 | 3-(4′-sulfobenzylidene)-bornan-2-one and its salts | 56039-58-8 |
| 27 | 4-tert-butyl-4′-methoxy-dibenzoylmethane | 70356-09-1 |
| 28 | 2,2′,4,4′-tetrahydroxybenzophenone | 131-55-5 |
| 29 | 2,2′-methylene-bis-[6-(2H-benzotriazol-2-yl)-4- | 103597-45-1 |
| | (1,1,3,3,-tetramethylbutyl)phenol] | |
| 30 | 2,2′-(1,4-phenylene)-bis-1H-benzimidazole-4, 6- | 180898-37-7 |
| | disulfonic acid, Na salt | |
| 31 | 2,4-bis-[4-(2-ethylhexyloxy)-2-hydroxy]phenyl- | 187393-00-6 |
| | 6-(4-methoxyphenyl)-(1,3,5)-triazine | |
| 32 | 3-(4-methylbenzylidene)-camphor | 36861-47-9 |
| 33 | 4-bis(polyethoxy)paraaminobenzoic | 113010-52-9 |
| | acid polyethoxyethyl ester | |
| 34 | 2,4-dihydroxybenzophenone | 131-56-6 |
| 35 | 2,2′-dihydroxy-4,4′-dimethoxybenzophenone-5,5′- | 3121-60-6 |
| | disodium-sulfonate | |
| 36 | 2-[4-(diethylamino)-2-hydroxybenzoyl]- | 302776-68-7 |
| | benzoic acid hexyl ester | |
| 37 | 2-(2H-benzotriazol-2-yl)-4-methyl-6- | 155633-54-8 |
| | [2-methyl-3-[1,3,3,3-tetramethyl-1- | |
| | [(trimethylsilyl)oxy]disiloxanyl]propyl]phenol | |
| 38 | 1,1-[(2,2′-dimethylpropoxy)carbonyl]- | 363602-15-7 |
| | 4,4-diphenyl-1,3-butadiene | |

In addition to the two abovementioned groups of primary sunscreen substances, secondary sunscreen agents of the antioxidant type, which interrupt the photochemical reaction chain triggered when UV radiation penetrates into the skin, can also be employed. Typical examples of these are superoxide dismutase, catalase, tocopherols (vitamin E) and ascorbic acid (vitamin C).

A further group are anti-irritants, which have an anti-inflammatory action on skin damaged by UV light. Such substances are, for example, bisabolol, phytol and phytantriol.

The list of active compounds and active compound combinations mentioned which can be used in the compositions according to the invention is of course not intended to be limiting. The active compounds can be used individually or in any desired combinations with one another.

The amount of such active compounds (one or more compounds) in the compositions according to the invention is preferably 0.001 to 30 wt. %, particularly preferably 0.05 to 20 wt. %, in particular 1 to 10 wt. %, based on the total weight of the composition.

The active compounds mentioned and further active compounds which can be used in the compositions according to the invention are mentioned in DE 103 18 526 A1 on pages 12 to 17, to which reference is made in its full scope at this point.

In the formulations according to the invention, the weight ratio of polymer molecularly imprinted with the active compound to active compound is in the range of from 1:10 to 100:1, preferably from 1:1 to 10:1, particularly preferably from 4:1 to 5:1 and in particular is 4:1.

### Fatty Phase

The formulations according to the invention include at least one fatty phase. Fatty phase is understood as meaning all cosmetically acceptable oils, fats and/or waxes.

### Constituents for Cosmetic Formulations According to the Invention

These constituents are preferably chosen from the group consisting of natural or synthetic polymers, pigments, humectants, oils, waxes, enzymes, minerals, vitamins, sunscreen agents, dyestuffs, fragrances, antioxidants, preservatives and/or pharmaceutical active compounds.

Suitable auxiliaries and additives for the preparation of skin cosmetic formulations are familiar to the person skilled in the art and can be found in cosmetics handbooks, for example Schrader, Grundlagen und Rezepturen der Kosmetika [Principles and Recipes of Cosmetics], Hüthig Verlag, Heidelberg, 1989, ISBN 3-7785-1491-1, or Umbach, Kosmetik: Entwicklung, Herstellung und Anwendung kosmetischer Mittel [Cosmetics: Development, Production and Use of Cosmetic Agents], 2nd expanded edition, 1995, Georg Thieme Verlag, ISBN 3 13 712602 9.

The polymers according to the invention which are molecularly imprinted with active compound are preferably used in dermocosmetics in combination with at least one constituent which differs therefrom and is chosen from cosmetically active compounds, emulsifiers, surfactants, preservatives, perfume oils, thickeners, hair polymers, hair and skin conditioners, graft polymers, water-soluble or dispersible silicone-containing polymers, sunscreen agents, bleaching agents, gelling agents, care agents, coloring agents, tinting agents, tanning agents, dyestuffs, pigments, agents for imparting consistency, humectants, re-oiling agents, collagen, protein hydrolysates, lipids, antioxidants, defoamers, antistatics, emollients and softeners.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, liponic acid and derivatives thereof (e.g. dihydroliponic acid), aurothioglucose, propyl-thiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) as well as salts thereof, dilauryl thiodipropionate, distearyl thiodipropionate, thiodipropionic acid and derivatives thereof (esters, ethers, peptides, lipids, nucleotides, nucleosides and salts) as well as sulfoximine compounds (e.g. buthionine sulfoximine, homocysteine sulfoximine, buthionine sulfones, penta-, hexa-, heptathionine sulfoximine) in very low tolerated dosages (e.g. pmol to pmol/kg), furthermore (metal) chelators, (e.g. α-hydroxy-fatty acids, palmitic acid, phytic acid, lactoferrin), α-hydroxy acids (e.g. citric acid, lactic acid, malic acid), humic acid, bile acid, bile extracts, bilirubin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives thereof (e.g. sodium ascorbate, ascorbyl palmitate, Mg ascorbyl phosphate, ascorbyl acetate), tocopherol and derivatives (e.g. vitamin E acetate, tocotrienol), vitamin A and derivatives (vitamin A palmitate) as well as coniferylbenzoate of benzoin resin, rutic acid and derivatives thereof, α-glycosylrutin, ferulic acid, furfurylidene, carnosine, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenium methionine) and stilbenes and derivatives thereof (e.g. stilbene oxide, trans-stilbene oxide)

The vitamins, provitamins or vitamin precursors of the vitamin B group or derivatives thereof and the derivatives of 2-furanone which are preferably to be employed according to the invention include, inter alia:

vitamin B₁, trivial name thiamine, chemical name 3-[(4′-amino-2′-methyl-5′-pyrimidinyl)methyl]-5-(2-hydroxyethyl)-4-methylthiazolium chloride,
vitamin B₂, trivial name riboflavin, chemical name 7,8-dimethyl-10-(1-D-ribityl)-benzo[g]pteridine-2,4(3H,10H)-dione. Riboflavin occurs e.g. in whey in the free form, and other riboflavin derivatives can be isolated from bacteria and yeasts. A stereoisomer of riboflavin which is likewise suitable according to the invention is the lyxoflavin which can be isolated from fish meal or liver and carries a D-arabityl radical instead of the D-ribityl radical.

Vitamin B₃. The compounds nicotinic acid and nicotinamide (niacinamide) are often mentioned by this name. Nicotinamide is preferred according to the invention.

Vitamin B₅ (pantothenic acid and panthenol). Panthenol is preferably employed. Derivatives of panthenol which can be employed according to the invention are, in particular, the esters and ethers of panthenol and cationically derivatized panthenols. In a further preferred embodiment of the invention, derivatives of 2-furanone can also be employed in addition to pantothenic acid or panthenol. Particularly preferred derivatives are the substances, which are also commercially obtainable, dihydro-3-hydroxy-4,4-dimethyl-2(3H)-furanone with the trivial name pantolactone (Merck), 4-hydroxymethyl-γ-butyrolactone (Merck), 3,3-dimethyl-2-hydroxy-γ-butyrolactone (Aldrich) and 2,5-dihydro-5-methoxy-2-furanone (Merck), all stereoisomers expressly being included.

These compounds advantageously impart to the dermocosmetics according to the invention moisture-donating and skin soothing properties.

Vitamin B₆, by which is understood not a uniform substance but the derivatives of 5-hydroxymethyl-2-methylpyridin-3-ol known by the trivial names pyridoxine, pyridoxamine and pyridoxol.

Vitamin B₇ (biotin), also called vitamin H or “skin vitamin”. Biotin is (3aS,4S,6aR)-2-oxohexahydrothienol[3,4-d]imidazole-4-valeric acid.

Panthenol, pantolactone, nicotinamide and biotin are very particularly preferred according to the invention.

### Dyestuffs

Dyestuffs which can be used are the substances which are suitable and approved for cosmetic purposes, such as are summarized, for example, in the publication “Kosmetische Färbemittel [Cosmetic Coloring Agents] of the Dyestuffs Commission of the Deutsche Forschungsgemeinschaft, published by Verlag Chemie, Weinheim, 1984. These dyestuffs are conventionally employed in a concentration of from 0.001 to 0.1 wt. %, based on the total mixture.

### Pigments

In a preferred embodiment, the compositions according to the invention comprise at least one pigment. The pigments are in the product mass in undissolved form and can be present in an amount of from 0.01 to 25 wt. %, particularly preferably from 5 to 15 wt. %. The preferred particle size is 1 to 200 μm, in particular 3 to 150 μm, particularly preferably 10 to 100 μm. The pigments are coloring agents which are practically insoluble in the use medium, and can be inorganic or organic. Inorganic-organic mixed pigments are also possible. Inorganic pigments are preferred. The advantage of the inorganic pigments is their excellent resistance to light, weather and heat. The inorganic pigments can be of natural origin, for example prepared from chalk, ochre, umber, green earth, calcined Terra di Siena or graphite. The pigments can be white pigments, such as e.g. titanium dioxide or zinc oxide, black pigments, such as e.g. iron oxide black, colored pigments, such as e.g. ultramarine or iron oxide red, iridescent pigments, metal effect pigments, pearlescent pigments and fluorescent or phosphorescent pigments, preferably at least one pigment being a colored, non-white pigment. Metal oxides, hydroxides and oxide hydrates, mixed phase pigments, sulfur-containing silicates, metal sulfides, complex metal cyanides, metal sulfates, chromates and molybdates and the metals themselves (bronze pigments) are suitable. Titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), iron blue (ferric ferrocyanide, CI 77510) and carmine (cochineal) are suitable in particular.

Pearlescent and colored pigments based on mica, which are coated with a metal oxide or a metal oxychloride, such as titanium dioxide or bismuth oxychloride, and optionally further color-imparting substances, such as iron oxides, iron blue, ultramarine, carmine etc., it being possible for the color to be determined by varying the layer thickness, are particularly preferred. Such pigments are marketed, for example, under the trade names Rona®, Colorona®, Dichrona® and Timiron® (Merck). Organic pigments are, for example, the natural pigments sepia, gamboges, bone charcoal, Cassel brown, indigo, chlorophyll and other plant pigments. Synthetic organic pigments are, for example, azo pigments, anthraquinoids, indigoids and dioxazine, quinacridone, phthalocyanine, isoindolinone, perylene and perinone, metal complex, alkali blue and diketopyrrolopyrrole pigments.

In one embodiment, the polymers according to the invention or prepared by the process according to the invention which are molecularly imprinted with active compound are used with at least one particulate substance, which is present in the composition in a content of from 0.01 to 10, preferably from 0.05 to 5 wt. %. Suitable substances are e.g. substances which are solid at room temperature (25° C.) and are in the form of particles. Silica, silicates, aluminates, aluminas, mica, salts, in particular inorganic metal salts, metal oxides, e.g. titanium dioxide, minerals and polymer particles, for example, are suitable. The particles are present in the composition in undissolved, preferably stably dispersed form and, after application to the use surface and evaporation of the solvent, can be deposited in the solid form. Preferred particulate substances are silica (silica gel, silicon dioxide) and metal salts, in particular inorganic metal salts, silica being particularly preferred. Metal salts are e.g. alkali metal or alkaline earth metal halides, such as sodium chloride or potassium chloride; and alkali metal or alkaline earth metal sulfates, such as sodium sulfate or magnesium sulfate.

### Pearlescent Agents

Possible pearlescent agents are, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which contain at least 24 carbon atoms in total, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Conventional thickeners in such formulations are crosslinked polyacrylic acids and derivatives thereof, polysaccharides and derivatives thereof, such as xanthan gum, agar-agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Nonionic thickeners are preferably employed.

Suitable cosmetically and/or dermocosmetically active compounds are e.g. coloring active compounds, skin and hair pigmenting agents, tinting agents, tanning agents, bleaching agents, keratin-hardening substances, antimicrobial active compounds, light filter active compounds, repellent active compounds, substances having a hyperemizing action, keratolytically and keratoplastically acting substances, antidandruff active compounds, antiphlogistics, substances having a keratinizing action, active compounds which are active antioxidatively or as agents which trap free radicals, skin moisturizing or moisture-retaining substances, re-oiling active compounds, antierythematously or antiallergically active compounds, branched fatty acids, such as 18-methyleicosanoic acid, and mixtures thereof.

Artificial skin-tanning active compounds which are suitable for tanning the skin without natural or artificial irradiation with UV rays are e.g. dihydroxyacetone, alloxan and walnut shell extract. Suitable keratin-hardening substances are as a rule active compounds such as are also employed in antiperspirants, such as e.g. potassium aluminum sulfate, aluminum hydroxychloride, aluminum lactate etc.

Antimicrobial active compounds are employed in order to destroy microorganisms or to inhibit their growth and thus serve both as preservatives and as a substance having a deodorizing action, which reduces the development or the intensity of body odor. These include e.g. conventional preservatives known to the person skilled in the art, such as p-hydroxybenzoic acid esters, imidazolidinyl-urea, formaldehyde, sorbic acid, benzoic acid, salicylic acid etc. Such substances having a deodorizing action are e.g. zinc ricinoleate, triclosan, undecylenoic acid alkylolamides, citric acid triethyl ester, chlorhexidine etc.

Suitable preservatives which are advantageously to be used according to the invention are:

TABLE 4
Suitable preservatives.

| | |
|---|---|
| E 200 | sorbic acid |
| E 201 | sodium sorbate |
| E 202 | potassium sorbate |
| E 203 | calcium sorbate |
| E 210 | benzoic acid |
| E 211 | sodium benzoate |
| E 212 | potassium benzoate |
| E 213 | calcium benzoate |
| E 214 | ethyl p-hydroxybenzoate |
| E 215 | ethyl p-hydroxybenzoate Na salt |
| E 216 | n-propyl p-hydroxybenzoate |
| E 217 | n-propyl p-hydroxybenzoate Na |
| | salt |
| E 218 | methyl p-hydroxybenzoate |
| E 219 | methyl p-hydroxybenzoate Na salt |
| E 220 | sulfur dioxide |
| E 221 | sodium sulfite |
| E 222 | sodium hydrogen sulfite |
| E 223 | sodium disulfite |
| E 224 | potassium disulfite |
| E 226 | calcium sulfite |
| E 227 | calcium hydrogen sulfite |
| E 228 | potassium hydrogen sulfite |
| E 230 | biphenyl (diphenyl) |
| E 231 | orthophenylphenol |
| E 232 | sodium |
| | orthophenylphenolate |
| E 233 | thiabendazole |
| E 235 | natamycin |
| E 236 | formic acid |
| E 237 | sodium formate |
| E 238 | calcium formate |
| E 239 | hexamethylenetetramine |
| E 249 | potassium nitrite |
| E 250 | sodium nitrite |
| E 251 | sodium nitrate |
| E 252 | potassium nitrate |
| E 280 | propionic acid |
| E 281 | sodium propionate |
| E 282 | calcium propionate |
| E 283 | potassium propionate |
| E 290 | carbon dioxide |

The E numbers listed in the above table are the usual designations in Directive 95/2/EEC.

Furthermore, preservatives or preservative auxiliaries which are usual in the cosmetics field, such as dibromodicyanobutane (2-bromo-2-bromomethylglutarodinitrile), 3-iodo-2-propynyl butyl carbamate, 2-bromo-2-nitro-propane-1,3-diol, imidazolidinylurea, 5-chloro-2-methyl-4-isothiazolin-3-one, 2-chloroacetamide, benzalkonium chloride and benzyl alcohol, are suitable according to the invention. Phenyl hydroxyalkyl ethers, in particular the compound known under the name phenoxyethanol, are furthermore suitable as preservatives on the basis of their bactericidal and fungicidal actions on a number of microorganisms.

Other germ-inhibiting agents are also likewise suitable for incorporation into the formulations according to the invention. Advantageous substances are, for example, 2,4,4′-trichloro-2′-hydroxydiphenyl ether (Irgasan), 1,6-di-(4-chloro-phenylbiguanido)-hexane (chlorhexidine), 3,4,4′-trichlorocarbanilide, quaternary ammonium compounds, clove oil, mint oil, thyme oil, triethyl citrate, farnesol (3,7,11-trimethyl-2,6,10-dodecatrien-1-ol) as well as the active compounds and active compound combinations described in the patent laid-open specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium bicarbonate is also advantageously to be used. Microbial polypeptides can likewise also be employed.

### Perfume Oils

The cosmetic compositions can optionally comprise perfume oils. Perfume oils which may be mentioned are, for example, mixtures of natural and synthetic odoriferous substances. Natural odoriferous substances are extracts from blossom (lily, lavender, rose, jasmine, orange-blossom, ylang-ylang), stems and leaves (geranium, patchouli, petitgrain), fruit (aniseed, coriander, caraway, juniper), fruit peel (bergamot, lemon, orange), roots (mace, angelica, celery, cardamom, costus, iris, calmus), wood (pine-, sandal-, guaiac-, cedar-, rosewood), herbs and grasses (tarragon, lemongrass, sage, thyme), needles and branches (spruce, fir, pine, dwarf pine) and resins and balsams (galbanum, elemi, benzoin, myrrh, frankincense, opoponax). Animal raw materials are furthermore possible, such as, for example, civet and castoreum. Typical synthetic odoriferous compounds are products of the ester, ether, aldehyde, ketone, alcohol and hydrocarbon type. Odoriferous compounds of the ester types are e.g. benzyl acetate, phenoxyethyl isobutyrate, 4-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl-propionate, styrallyl propionate and benzyl salicylate. The ethers include, for example, benzyl ethyl ether, the aldehydes include e.g. the alkanals having 8 to 18 carbon atoms, citral, citronellal, citronellyloxyacetaldehyde, cyclamenaldehyde, hydroxycitronellal, lilial and bourgeonat, the ketones include e.g. the ionones, cc-isomethylionene and methyl cedryl ketone, the alcohols include anethole, citronellol, eugenol, isoeugenol, geraniol, linalool, phenylethyl alcohol and terpineol, and the hydrocarbons include chiefly the terpenes and balsams. Preferably, however, mixtures of various odoriferous substances which together generate a pleasant fragrance are used. Essential oils of relatively low volatility, which are usually used as aroma components, are also suitable as perfume oils, e.g. sage oil, chamomile oil, clove oil, Melissa oil, mint oil, cinnamon leaf oil, linden blossom oil, juniper berry oil, vetiver oil, frankincense oil, galbanum oil, labolanum oil and lavandin oil. Bergamot oil, dihydromyrcenol, lilial, lyral, citronellol, phenylethyl alcohol, α-hexylcinnamaldehyde, geraniol, benzylacetone, cyclamenaldehyde, linalool, Boisambrene® Forte, ambroxan, indole, hedione, sandelice, lemon oil, mandarin oil, orange oil, allyl amylglycolate, cyclovertal, lavandin oil, clary sage oil, β-damascone, Bourbon geranium oil, cyclohexyl salicylate, Vertofix® Coeur, Iso-E-Super®, Fixolide® NP, evernyl, iraldein gamma, phenylacetic acid, geranyl acetate, benzyl acetate, rose oxide, romillate, irotyl and floramate, by themselves or in mixtures, are preferably employed.

### Oils, Fats and Waxes

The compositions according to the invention comprise at least one fatty phase. Fatty phase is understood as meaning oils, fats and/or waxes. Constituents of the oily and/or fatty phase of the compositions according to the invention are advantageously chosen from the group consisting of lecithins and fatty acid triglycerides, namely the triglycerol esters of saturated and/or unsaturated, branched and/or unbranched alkanecarboxylic acids of a chain length of 8 to 24, in particular 12 to 18 C atoms. The fatty acid triglycerides can advantageously be chosen, for example, from the group consisting of synthetic, semi-synthetic and naturally occurring oils, such as e.g. olive oil, sunflower oil, soya oil, groundnut oil, rape oil, almond oil, palm oil, coconut oil, castor oil, wheat germ oil, grape seed oil, thistle oil, evening primrose oil, macadamia nut oil and many of the like. Further polar oil components can be chosen from the group consisting of esters of saturated and/or unsaturated, branched and/or unbranched alkanecarboxylic acids of a chain length of from 3 to 30 C atoms and saturated and/or unsaturated, branched and/or unbranched alcohols of a chain length of from 3 to 30 C atoms and from the group consisting of esters of aromatic carboxylic acids and saturated and/or unsaturated, branched and/or unbranched alcohols of a chain length of from 3 to 30 C atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl iso-nonanoate, 2-ethylhexyl palmitate, 2-ethyl hexyl laurate, 2-hexyldecyl stearate, 2-octyldodecyl palmitate, oleyl oleate, oleyl erucate, erucyl oleate, erucyl erucate, dicaprylyl carbonate (Cetiol CC) and cocoglycerides (Myritol 331), butylene glycol dicaprylate/dicaprate and dibutyl adipate, and synthetic, semi-synthetic and natural mixtures of such esters, such as e.g. jojoba oil.

One or more oil components can furthermore advantageously be chosen from the group consisting of branched and unbranched hydrocarbons and hydrocarbon waxes, silicone oils, dialkyl ethers and the group consisting of saturated or unsaturated, branched or unbranched alcohols. Any desired blends of such oil and wax components are also advantageously to be employed in the context of the present invention. It may also optionally be advantageous to employ waxes, for example cetyl palmitate, as the sole lipid component of the oily phase. According to the invention, the oily component is advantageously chosen from the group consisting of 2-ethylhexyl isostearate, octyldodecanol, isotridecyl isononanoate, isoeicosane, 2-ethylhexyl cocoate, C12-15-alkyl benzoate, caprylic/capric acid triglyceride and dicaprylyl ether. Mixtures of C12-15-alkyl benzoate and 2-ethylhexyl isostearate, mixtures of C12-15-alkyl benzoate and isotridecyl isononanoate as well as mixtures of C12-15-alkyl benzoate, 2-ethylhexyl isostearate and isotridecyl isononanoate are advantageous according to the invention. According to the invention, fatty acid triglycerides, in particular soya oil and/or almond oil, are particularly preferably employed as oils having a polarity of from 5 to 50 mN/m. Of the hydrocarbons, paraffin oil, squalane and squalene are advantageously to be used in the context of the present invention.

The oily phase can furthermore advantageously be chosen from the group consisting of Guerbet alcohols. Guerbet alcohols are named after Marcel Guerbet, who described their preparation for the first time. They are formed in accordance with the equation

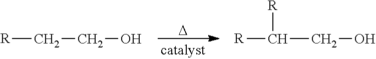

by oxidation of an alcohol to an aldehyde, by aldol condensation of the aldehyde, splitting off of water from the aldol and hydrogenation of the allylaldehyde. Guerbet alcohols are liquid even at relatively low temperatures and cause practically no irritation to skin. They can advantageously be employed as constituents having an oiling, super-oiling and also re-oiling action in cosmetic compositions.

The use of Guerbet alcohols in cosmetics is known per se. Such species are in this case usually distinguished by the structure:

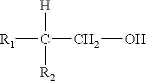

In this, R₁ and R₂ as a rule denote unbranched alkyl radicals.

According to the invention, the Guerbet alcohol or alcohols are advantageously chosen from the group wherein

R₁=propyl, butyl, pentyl, hexyl, heptyl or octyl and
R₂=hexyl, heptyl, octyl, nonyl, decyl, undecyl, dodecyl, tridecyl or tetradecyl.

Guerbet alcohols which are preferred according to the invention are 2-butyloctanol (for example commercially obtainable as Isofol® 12 (Condea)) and 2-hexyldecanol (for example commercially obtainable as Isofol® 16 (Condea)). Mixtures of Guerbet alcohols according to the invention are also advantageously to be used according to the invention, such as, for example, mixtures of 2-butyloctanol and 2-hexyldecanol (for example commercially obtainable as Isofol® 14 (Condea)).

Any desired blends of such oil and wax components are also advantageously to be employed in the context of the present invention. Among the polyolefins, polydecenes are the preferred substances.

The oil component can furthermore advantageously have a content of cyclic or linear silicone oils or consist entirely of such oils, although it is preferable to use an additional content of other oily phase components in addition to the silicone oil or the silicone oils. Low molecular weight silicones or silicone oils are as a rule defined by the following general formula:

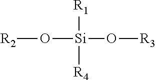

Higher molecular weight silicones or silicone oils are as a rule defined by the following general formula:

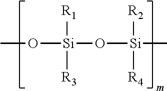

wherein the silicon atoms can be substituted by identical or different alkyl radicals and/or aryl radicals, which are represented here generally by the radicals R₁ to R₄. However, the number of different radicals is not necessarily limited to up to 4. m can assume values here of from 2 to 200,000.

Cyclic silicones which are advantageously to be employed according to the invention are as a rule defined by the following general formula

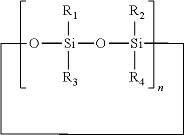

wherein the silicon atoms can be substituted by identical or different alkyl radicals and/or aryl radicals, which are represented here generally by the radicals R₁ to R₄. However, the number of different radicals is not necessarily limited to up to 4. “n” can assume values of from 3/2 to 20 here. Fraction values for n take into account that uneven number of siloxy groups can be present in the ring.

Phenyltrimethicone is advantageously chosen as the silicone oil. Other silicone oils, for example dimethicone, hexamethylcyclotrisiloxane, phenyldimethicone, cyclomethicone (octamethylcyclotetrasiloxane), hexamethylcyclotrisiloxane, polydimethylsiloxane, poly(methylphenylsiloxane), cetyldimethicone and behenoxydimethicone, are also advantageously to be used in the context of the present invention. Mixtures of cyclomethicone and isotridecyl isononanoate, and those of cyclomethicone and 2-ethylhexyl isostearate are furthermore advantageous. However, it is also advantageous to choose silicone oils of a structure similar to the compounds described above, the organic side chains of which are derivatized, for example polyethoxylated and/or polypropoxylated. These include, for example, polysiloxane-polyalkyl polyether copolymers, such as e.g. cetyl-dimethicone copolyol. Cyclomethicone (octamethylcyclotetrasiloxane) is advantageously employed as the silicone oil to be used according to the invention. Fat and/or wax components which are advantageously to be used according to the invention can be chosen from the group consisting of plant waxes, animal waxes, mineral waxes and petrochemical waxes. Waxes which are advantageous are, for example, candelilla wax, carnauba wax, Japan wax. esparto grass wax, cork wax, guarama wax, rice germ oil wax, sugar cane wax, berry wax, ouricury wax, montan wax, jojoba wax, shea butter, beeswax, shellac wax, spermaceti, lanolin (wool wax), uropygium fat, ceresin, ozocerite (earth wax), paraffin waxes and microwaxes.

Further advantageous fat and/or wax components are chemically modified waxes and synthetic waxes, such as, for example, Syncrowax® HRC (glyceryl tribehenate) and Syncrowax® AW 1 C (C₁₈₋₃₆-fatty acid) and montan ester waxes, Sasol waxes, hydrogenated jojoba waxes, synthetic or modified beeswaxes (e.g. dimethicone copolyol beeswax and/or C₃₀₋₅₀-alkyl-beeswax), cetyl ricinoleates, such as, for example, Tegosoft® CR, polyalkylene waxes, polyethylene glycol waxes, but also chemically modified fats, such as e.g. hydrogenated plant oils (for example hydrogenated castor oil and/or hydrogenated coco-fatty glycerides), triglycerides, such as, for example, soya glycerides, trihydroxystearin, fatty acids, fatty acid esters and glycol esters, such as, for example, C₂₀₋₄₀-alkyl stearate, C₂₀₋₄₀-alkyl hydroxystearoylstearate and/or glycol montanate. Certain organosilicon compounds which have physical properties similar to the fat and/or wax components mentioned, such as, for example, stearoxytrimethylsilanes, are furthermore also advantageous.

According to the invention, the fat and/or wax components can be used in the compositions either individually or as a mixture. Any desired blends of such oil and wax components are also advantageously to be employed in the context of the present invention. The oily phase is advantageously chosen from the group consisting of 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic/capric acid triglyceride and dicaprylyl ether. Mixtures of octyl-dodecanol, caprylic-capric acid triglyceride, dicaprylyl ether, dicaprylyl carbonate, coco-glycerides or mixtures of C₁₂₋₁₅-alkyl benzoate and 2-ethyl-hexyl isostearate, mixtures of C₁₂₋₁₅-alkyl benzoate and butylene glycol dicaprylate/dicaprate as well as mixtures of C₁₂₋₁₅-alkyl benzoate, 2-ethylhexyl isostearate and isotridecyl isononanoate are particularly advantageous. Of the hydrocarbons, paraffin oil, cycloparaffin, squalane, squalene, hydrogenated polyisobutene and polydecene are advantageously to be used in the context of the present invention.

The oil component is furthermore advantageously chosen from the group consisting of phospholipids. The phospholipids are phosphoric acid esters of acylated glycerols. The lecithins, for example, which are distinguished by the general structure

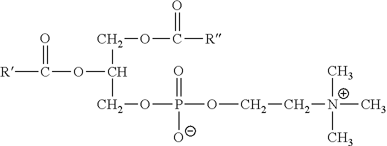

wherein R′ and R″ typically represent unbranched aliphatic radicals having 15 or 17 carbon atoms and up to 4 cis double bonds, are of the greatest importance among the phosphatidylcholines.

According to the invention, Merkur White Oil Pharma 40 from Merkur Vaseline, Shell Ondina® 917, Shell Ondina® 927, Shell Oil 4222 and Shell Ondina® 933 from Shell & DEA Oil, and Pionier® 6301 S and Pionier® 2071 (Hansen & Rosenthal) can be employed as paraffin oil which is advantageous according to the invention. Suitable cosmetically acceptable oil and fat components are described in Karl-Heinz Schrader, Grundlagen and Rezepturen der Kosmetika [Principles and Recipes of Cosmetics], 2nd edition, Verlag Hüthig, Heidelberg, p. 319-355, to which reference is made here in its full scope.

### Solvents

If the polymers according to the invention or prepared by the process according to the invention which are molecularly imprinted with active compound are used in cosmetic or dermatological formulations which are a solution or emulsion or dispersion, solvents which can be used are:

water or aqueous solutions; oils, such as triglycerides of capric or of caprylic acid, but preferably castor oil; fats, waxes and other natural and synthetic fatty substances, preferably esters of fatty acids with alcohols of low C number, e.g. with isopropanol, propylene glycol or glycerol, or esters of fatty alcohols with alkanoic acids of low C number or with fatty acids; alcohols, diols or polyols of low C number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products. Mixtures of the abovementioned solvents can be used in particular. In the case of alcoholic solvents, water can be a further constituent.

### Surfactants

According to the invention, in addition to the polymers according to the invention or prepared by the process according to the invention which are molecularly imprinted with active compound, compositions can also comprise surfactants. Such surfactants are, for example:
phosphoric acid esters and salts, such as, for example, DEA-oleth-10 phosphate and dilaureth-4 phosphate,
alkyl-sulfonates, for example sodium coco-monoglyceride sulfate, sodium C₁₂₋₁₄-olefin-sulfonate, sodium lauryl sulfoacetate and magnesium PEG-3 cocamidosulfate,
carboxylic acids and derivatives, such as, for example, lauric acid, aluminum stearate, magnesium alkanolate and zinc undecylenate, and ester-carboxylic acids, for example calcium stearoyl-lactylate, laureth-6 citrate and sodium PEG-4 lauramidocarboxylate,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers and alkyl polyglycosides, such as lauryl glucoside, decyl glycoside and coco-glycoside.

### Polysorbates

According to the invention, in addition to the polymers according to the invention or prepared by the process according to the invention which are molecularly imprinted with active compound, the compositions can also comprise polysorbates.

Polysorbates which are advantageous here in the context of the invention are
polyoxyethylene(20) sorbitan monolaurate (Tween 20, CAS no. 9005-64-5)
polyoxyethylene(4) sorbitan monolaurate (Tween 21, CAS no. 9005-64-5)
polyoxyethylene(4) sorbitan monostearate (Tween 61, CAS no. 9005-67-8)
polyoxyethylene(20) sorbitan tristearate (Tween 65, CAS no. 9005-71-4)
polyoxyethylene(20) sorbitan monooleate (Tween 80, CAS no. 9005-65-6)
polyoxyethylene(5) sorbitan monooleate (Tween 81, CAS no. 9005-65-5)
polyoxyethylene(20) sorbitan triooleate (Tween 85, CAS no. 9005-70-3).

Particularly advantageous polysorbates are, in particular,
polyoxyethylene(20) sorbitan monopalmitate (Tween 40, CAS no. 9005-66-7)
polyoxyethylene(20) sorbitan monostearate (Tween 60, CAS no. 9005-67-8)

These are advantageously employed according to the invention in a concentration of from 0.1 to 5% by weight, and in particular in a concentration of from 1.5 to 2.5% by weight, based on the total weight of the composition, individually or as a mixture of several polysorbates.

### Conditioning Agents

In a preferred embodiment of the invention, the compositions also comprise conditioning agents. Conditioning agents which are preferred according to the invention are, for example, all the compounds which are listed in the International Cosmetic Ingredient Dictionary and Handbook (volume 4, editors: R. C. Pepe, J. A. Wenninger, G. N. McEwen, The Cosmetic, Toiletry and Fragrance Association, 9th edition, 2002) under Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectans, and all the compounds listed in EP-A 934 956 (p. 11-13) under “water soluble conditioning agent” and “oil soluble conditioning agent”. Further advantageous conditioning agents are, for example, the compounds called polyquaternium in accordance with the INCI (in particular polyquaternium-1 to polyquaternium-56).

Suitable conditioning agents also include, for example, polymeric quaternary ammonium compounds, cationic cellulose derivatives and polysaccharides.

In this context, conditioning agents which are advantageous according to the invention can be chosen from among the compounds shown in the following table.

TABLE 5
Conditioning agents which are advantageously to be used

| | | | Example |
|---|---|---|---|
| INCl name | CAS number | Polymer type | (trade name) |
| Poly- | CAS 63451-27-4 | urea, N,N′,-bis[3- | Mirapol ® A- |
| quaternium-2 | | (dimethylamino)propyl]-polymer with | 15 |
| | | 1,1′-oxybis(2-chloroethane) | |
| Poly- | CAS 26006-22-4 | acrylamide, β- | |
| quaternium-5 | | methacryloxyethyltriethyl-ammonium | |
| | | methosulfate | |
| Poly- | CAS 26062-79-3 | N,N-dimethyl-N-2-propenyl-2- | Merquat ® |
| quaternium-6 | | propenaminium chloride | 100 |
| Poly- | CAS 26590-05-6 | N,N-dimethyl-N-2-propenyl-2- | Merquat ® S |
| quaternium-7 | | propenaminium chloride, 2- | |
| | | propenamide | |
| Poly- | CAS 53568-66-4, | quaternary ammonium salt of | Celquat ® |
| quaternium-10 | 55353-19-0, 54351- | hydroxyethylcellulose | SC-230M, |
| | 50-7, 68610-92-4, | | Polymer JR |
| | 81859-24-7 | | 400 |
| Poly- | CAS 53633-54-8 | vinylpyrrolidone/dimethylaminoethyl | Gafquat ® |
| quaternium-11 | | methacrylate copolymer/diethyl sulfate | 755N |
| | | reaction product | |
| Poly- | CAS 29297-55-0 | vinylpyrrolidone/vinylimidazolinium | Luviquat ® |
| quaternium-16 | | methochloride copolymer | HM552 |
| Poly- | CAS 90624-75-2 | | Mirapol ® |
| quaternium-17 | | | AD-1 |
| Poly- | CAS 110736-85-1 | quaternized water-soluble polyvinyl | |
| quaternium-19 | | alcohol | |
| Poly- | CAS 110736-86-2 | water-dispersible quaternized polyvinyl | |
| quaternium-20 | | octadecyl ether | |
| Poly- | | polysiloxane/polydimethyl- | Abil ® B 9905 |
| quaternium-21 | | dimethylammonium acetate copolymer | |
| Poly- | CAS 53694-17-0 | dimethyldiallylammonium | Merquat ® |
| quaternium-22 | | chloride/acrylic acid copolymer | 280 |
| Poly- | CAS 107897-23-5 | polymeric quaternary ammonium salt | Quartisoft ® |
| quaternium-24 | | of hydroxyethylcellulose | LM-200 |
| Poly- | CAS 131954-48-8 | vinylpyrrolidone/methacryl- | Gafquat ® |
| quaternium-28 | | amidopropyltrimethylammonium | HS-100 |
| | | chloride copolymer | |
| Poly- | CAS 92091-36-6, | chitosan which has been reacted with | Lexquat ® |
| quaternium-29 | 148880-30-2 | propylene oxide and quaternized with | CH |
| | | epichlorohydrin | |
| Poly- | CAS 136505-02-7, | polymeric, quaternary ammonium salt | Hypan ® QT |
| quaternium-31 | 139767-67-7 | prepared by reaction of DMAPA | 100 |
| | | acrylate/acrylic acid/acrylonitrogen | |
| | | copolymers and diethyl sulfate | |
| Poly- | CAS 35429-19-7 | N,N,N-trimethyl-2-([82-methyl-1-oxo-2- | |
| quaternium-32 | | propenyl)oxy]-ethanaminium chloride, | |
| | | polymer with 2-propenamide | |
| Poly- | CAS 26161-33-1 | | |
| quaternium-37 | | | |
| Poly- | | copolymeric quaternary ammonium | |
| quaternium-44 | | salt of vinylpyrrolidone and | |
| | | quaternized imidazoline | |

Further conditioning agents which are advantageous according to the invention are cellulose derivatives and quaternized guar gum derivatives, in particular guar-hydroxypropylammonium chloride (e.g. Jaguar Excel® and Jaguar C 162® (Rhodia), CAS 65497-29-2, CAS 39421-75-5).

Nonionic poly-N-vinyl pyrrolidone/polyvinyl acetate copolymers (e.g. Luvisko® VA 64 (BASF Aktiengesellschaft)), anionic acrylate copolymers (e.g. Luviflex® Soft (BASF Aktiengesellschaft)), and/or amphoteric amide/acrylate/methacrylate copolymers (e.g. Amphomer® (National Starch)) can also advantageously be employed according to the invention as conditioning agents.

### Powder Raw Materials

An addition of powder raw materials can generally be advantageous. The use of talc is particularly preferred.

### Ethoxylated Glycerol Fatty Acid Esters

According to the invention, in addition to the molecularly imprinted polymers the compositions can also comprise ethoxylated oils chosen from the group consisting of ethoxylated glycerol fatty acid esters, particularly preferably PEG-10 olive oil glycerides, PEG-11 avocado oil glycerides, PEG-11 cacao butter glycerides, PEG-13 sunflower oil glycerides, PEG-15 glyceryl isostearate, PEG-9 coco-fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coco-fatty acid glycerides, PEG-7 glyceryl cocoate, PEG-45 palm kernel oil glycerides, PEG-35 castor oil, olive oil PEG-7 ester, PEG-6 caprylic acid/capric acid glycerides, PEG-10 olive oil glycerides, PEG-13 sunflower oil glycerides, PEG-7 hydrogenated castor oil, hydrogenated palm kernel oil glyceride PEG-6 eater, PEG-20 maize oil glycerides, PEG-18 glyceryl oleate-cocoate, PEG-40 hydrogenated castor oil, PEG-40 castor oil, PEG-60 hydrogenated castor oil, PEG-60 maize oil glycerides, PEG-54 hydrogenated castor oil, PEG-45 palm kernel oil glycerides, PEG-35 castor oil, PEG-80 glyceryl cocoate, PEG-60 almond oil glycerides, PEG-60 “evening primrose” glycerides, PEG-200 hydrogenated glyceryl palmate and PEG-90 glyceryl isostearate.

Preferred ethoxylated oils are PEG-7 glyceryl cocoate, PEG-9 coco-glycerides, PEG-40 hydrogenated castor oil and PEG-200 hydrogenated glyceryl palmate. Ethoxylated glycerol fatty acid esters are employed for various purposes in aqueous cleansing recipes. Glycerol fatty acid esters having a low degree of ethoxylation (3-12 ethylene oxide units) conventionally serves as re-oiling agents for improving the sensation on the skin after drying out, and glycerol fatty acid esters having a degree of ethoxylation of approx. 30-50 serve as solubilizing agents for non-polar substances, such as perfume oils. Highly ethoxylated glycerol fatty acid esters are employed as thickeners. All these substances have the common feature that they generate a particular sensation on the skin when used when diluted with water.

### Sunscreen Agents

The invention also provides the use of the polymers according to the invention or prepared by the process according to the invention which are molecularly imprinted with active compound in dermocosmetic formulations in combination with sunscreen agents. These cosmetic and/or dermatological sunscreen compositions serve for cosmetic and/or dermatological protection from light, and furthermore for treatment and care of the skin and/or hair and as a make-up product in decorative cosmetics. They include, for example, sun creams, lotions, milks, oils, balsams and gels, lip care products and lipsticks, covering creams and sticks, moisturizing creams, lotions and emulsion, face, body and hand creams, hair treatments and conditioners, hair setting compositions, styling gels, hair sprays, roll-on deodorants or eye wrinkle creams, tropicals, sunblocks and after-sun preparations. All the preparations comprise at least one polymer molecularly imprinted with active compound and one of the UV filter substances mentioned.

Sun oils are usually mixture of various oils with one or more sunscreen filters and perfume oils. The oil components are chosen according to various cosmetic properties. Oils which oil well and impart a soft skin sensation, such as mineral oils (e.g. paraffin oils) and fatty acid triglycerides (e.g. groundnut oil, sesame oil, avocado oil, medium-chain triglycerides), are mixed with oils which improve the ease of distribution and the absorption of the sun oils into the skin, reduce the tackiness and make the oil film permeable to air and water vapor (perspiration). These include branched-chain fatty acid esters (e.g. isopropyl palmitate) and silicone oils (e.g. dimethylsilicone). If oils based on unsaturated fatty acids are used, antioxidants, e.g. tocopherol, are added in order to prevent them from becoming rancid. As water-free formulations, sun oils as a rule contain no preservatives. Sun milk and creams are prepared as oil-in-water (O/W) emulsions and as water-in-oil (W/O) emulsions. The properties of the preparations are very different, depending on the emulsion type. O/W emulsions can be easily distributed over the skin, they are usually absorbed rapidly and almost always can be washed off easily with water. W/O emulsions are more difficult to rub in, they oil the skin more intensively and as a result have a somewhat tackier effect, but on the other hand protect the skin better from drying out. W/O emulsions are usually water-resistant. In the case of O/W emulsions, the degree of water resistance is decided by the emulsion base, the choice of suitable sunscreen substances and, where appropriate, the use of auxiliaries (e.g. polymers). The bases of liquid and cream-like O/W emulsions are similar in composition to the emulsions otherwise conventional in skin care. Sun milks should oil sufficiently skin which has been dried out by sun, water and wind. They should not be tacky, since this is felt as particularly unpleasant in the heat and in contact with sand. The sunscreen compositions are as a rule based on a carrier, which contains at least an oily phase. However, compositions based solely on water are also possible. Accordingly, oils, oil-in-water and water-in-oil emulsions, creams and pastes, lip protection stick compositions or fat-free gels are possible. Possible emulsions are, inter alia, also O/W macroemulsions, O/W microemulsions or O/W/O emulsions with surface-coated titanium dioxide particles present in dispersed form, the emulsions being obtainable by phase inversion technology, in accordance with DE-A-197 26 121.

Conventional cosmetic auxiliaries which are possible additives are e.g. (co)emulsifiers, fats and waxes, stabilizers, thickeners, biogenic active compounds, film-forming agents, fragrances, dyestuffs, pearlescent agents, preservatives, pigments, electrolytes (e.g. magnesium sulfate) and pH regulators. Stabilizers which can be employed are metal salts of fatty acids, such as e.g. magnesium, aluminum and/or zinc stearate. Biogenic active compounds are to be understood as meaning, for example, plant extracts, protein hydrolysates and vitamin complexes. The usual film-forming agents are, for example, hydrocolloids, such as chitosan, microcrystalline chitosan or quaternized chitosan, polyvinylpyrrolidone, vinylpyrrolidone/vinyl acetate copolymers, polymers of the acrylic acid series, quaternary cellulose derivatives and similar compounds.

Suitable light filter active compounds are substances which absorb UV rays in the UV-B and/or UV-A range. By these there are to be understood organic substances which are capable of absorbing ultraviolet rays and of releasing the energy absorbed again in the form of longer-wavelength radiation, e.g. heat. The organic substances can be oil-soluble or water-soluble. Suitable UV filters are e.g. 2,4,6-triaryl-1,3,5-triazines, in which the aryl groups in each case can carry at least one substituent, which is preferably chosen from hydroxyl, alkoxy, specifically methoxy, and alkoxycarbonyl, specifically methoxycarbonyl and ethoxycarbonyl. p-Aminobenzoic acid esters, cinnamic acid esters, benzophenones, camphor derivatives and pigments which keep out UV rays, such as titanium dioxide, talc and zinc oxide, are furthermore suitable. Pigments based on titanium dioxide are particularly preferred.

Oil-soluble UV-B filters which can be used are e.g. the following substances:

3-benzylidenecamphor and derivatives thereof, e.g. 3-(4-methylbenzylidene)camphor;
4-aminobenzoic acid derivatives, preferably 4-(dimethylamino)benzoic acid 2-ethylhexyl ester, 4-(dimethylamino)benzoic acid 2-octyl ester and 4-(dimethylamino)benzoic acid amyl ester;
esters of cinnamic acid, preferably 4-methoxycinnamic acid 2-ethylhexyl ester, 4-methoxycinnamic acid propyl ester, 4-methoxycinnamic acid isoamyl ester, 4-methoxycinnamic acid isopentyl ester and 2-cyano-3-phenyl-cinnamic acid 2-ethylhexyl ester (octocrylene);
esters of salicylic acid, preferably salicylic acid 2-ethylhexyl ester, salicylic acid 4-isopropylbenzyl ester and salicylic acid homomethyl ester;
derivatives of benzophenone, preferably 2-hydroxy-4-methoxybenzophenone, 2-hydroxy-4-methoxy-4′-methyl benzophenone and 2,2′-dihydroxy-4-methoxy-benzophenone;
esters of benzalmalonic acid, preferably 4-methoxybenzalmalonic acid di-2-ethylhexyl ester;
triazine derivatives, such as e.g. 2,4,6-trianilino-(p-carbo-2′-ethyl-1′-hexyloxy)-1,3,5-triazine (octyltriazone) and dioctyl butamido triazone (Uvasorb® HEB);
propane-1,3-diones, such as e.g. 1-(4-tert-butylphenyl)-3-(4′-methoxyphenyl)propane-1,3-dione.

Possible water-soluble substances are:

2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and its salts;
sulphonic acid derivatives of 3-benzylidenecamphor, such as e.g. 4-(2-oxo-3-bornylidenemethyl)benzenesulphonic acid and 2-methyl-5-(2-oxo-3-bornylidene)sulphonic acid and salts thereof.

The use of esters of cinnamic acid, preferably 4-methoxycinnamic acid 2-ethylhexyl ester, 4-methoxycinnamic acid isopentyl ester and 2-cyano-3-phenyl-cinnamic acid 2-ethylhexyl ester (octocrylene), is particularly preferred.

The use of derivatives of benzophenone, in particular 2-hydroxy-4-methoxybenzophenone, 2-hydroxy-4-methoxy-4′-methylbenzophenone and 2,2′-dihydroxy-4-methoxybenzophenone, and the use of propane-1,3-diones, such as e.g. 1-(4-tert-butylphenyl)-3-(4′-methoxyphenyl)propane-1,3-dione, is furthermore preferred.

Possible typical UV-A filters are:

derivatives of benzoylmethane, such as, for example, 1-(4′-tert-butylphenyl)-3-(4′-methoxyphenyl)-propane-1,3-dione, 4-tert-butyl-4′-methoxydibenzoylmethane or 1-phenyl-3-(4′-isopropylphenyl)-propane-1,3-dione;
amino-hydroxy-substituted derivatives of benzophenones, such as e.g. N,N-diethylamino-hydroxybenzoyl-n-hexyl-benzoate.

The UV-A and UV-B can of course also be employed in mixtures.

Further suitable UV filter substances are mentioned in the following table.

TABLE 6
Suitable sunscreen agents

| | | CAS no. |
|---|---|---|
| No. | Substance | (= acid) |
| 1 | 4-aminobenzoic acid | 150-13-0 |
| 2 | 3-(4′-trinnethylammonium)-benzylidenebornan- | 52793-97-2 |
| | 2-one methyl-sulfate | |
| 3 | 3,3,5-trimethyl-cyclohexyl salicylate | 118-56-9 |
| | (homosalatum) | |
| 4 | 2-hydroxy-4-methoxy-benzophenone | 131-57-7 |
| | (oxybenzonum) | |
| 5 | 2-phenylbenzimidazole-5-sulfonic acid and its | 27503-81-7 |
| | potassium, sodium and triethanolamine salts | |
| 6 | 3,3′-(1,4-phenylenedimethine)-bis(7,7-dimethyl-2- | 90457-82-2 |
| | oxobicyclo[2.2.1]heptane-1-methanesulfonic acid) | |
| | and its salts | |
| 7 | 4-bis(polyethoxy)amino-benzoic acid | 113010-52-9 |
| | polyethoxy-ethyl ester | |
| 8 | 4-dimethylamino-benzoic acid 2-ethylhexyl ester | 21245-02-3 |
| 9 | salicylic acid 2-ethylhexyl ester | 118-60-5 |
| 10 | 4-methoxy-cinnamic acid 2-isoamyl ester | 71617-10-2 |
| 11 | 4-methoxy-cinnamic acid 2-ethylhexyl ester | 5466-77-3 |
| 12 | 2-hydroxy-4-methoxy-benzophenone-5-sulfonic | 4065-45-6 |
| | acid (sulisobenzonum) and the sodium salt | |
| 13 | 3-(4′-sulfobenzylidene)-bornan-2-one and salts | 58030-58-6 |
| 14 | 3-benzylidenebornan-2-one | 16087-24-8 |
| 15 | 1-(4′-isopropylphenyl)-3-phenylpropane-1,3-dione | 63260-25-9 |
| 16 | 4-isopropylbenzyl salicylate | 94134-93-7 |
| 17 | 3-imidazol-4-yl-acrylic acid and its ethyl ester | 104-98-3 |
| 18 | 2-cyano-3,3-diphenylacrylic acid ethyl ester | 5232-99-5 |
| 19 | 2-cyano-3,3-diphenylacrylic acid 2′-ethylhexyl ester | 6197-30-4 |
| 20 | menthyl o-aminobenzoate or: | 134-09-8 |
| | 5-methyl-2-(1-methylethyl)-2-aminobenzoate | |
| 21 | glyceryl p-aminobenzoate or: | 136-44-7 |
| | 4-aminobenzoic acid 1-glyceryl ester | |
| 22 | 2,2′-dihydroxy-4-methoxybenzophenone | 131-53-3 |
| | (dioxybenzone) | |
| 23 | 2-hydroxy-4-methoxy-4-methyl benzophenone | 1641-17-4 |
| | (mexenone) | |
| 24 | triethanolamine salicylate | 2174-16-5 |
| 25 | dimethoxyphenylglyoxalic acid or: | 4732-70-1 |
| | sodium 3,4-dimethoxy-phenyl-glyoxalate | |
| 26 | 3-(4′-sulfobenzylidene)-bornan-2-one and its salts | 56039-58-8 |
| 27 | 4-tert-butyl-4′-methoxy-dibenzoylmethane | 70356-09-1 |
| 28 | 2,2′,4,4′-tetrahydroxybenzophenone | 131-55-5 |
| 29 | 2,2′-methylene-bis-[6-(2H-benzotriazol-2-yl)-4- | 103597-45-1 |
| | (1,1,3,3,-tetramethylbutyl)phenol] | |
| 30 | 2,2′-(1,4-phenylene)-bis-1H-benzimidazole-4,6- | 180898-37-7 |
| | disulfonic acid, Na salt | |
| 31 | 2,4-bis-[4-(2-ethylhexyloxy)-2-hydroxy]phenyl- | 187393-00-6 |
| | 6-(4-methoxyphenyl)-(1,3,5)-triazine | |
| 32 | 3-(4-methylbenzylidene)-camphor | 36861-47-9 |
| 33 | 4-bis(polyethoxy)paraaminobenzoic acid | 113010-52-9 |
| | polyethoxyethyl ester | |
| 34 | 2,4-dihydroxybenzophenone | 131-56-6 |
| 35 | 2,2′-dihydroxy-4,4′-dimethoxybenzophenone-5,5′- | 3121-60-6 |
| | disodium-sulfonate | |
| 36 | 2-[4-(diethylamino)-2-hydroxybenzoyl]- | 302776-68-7 |
| | benzoic acid hexyl ester | |
| 37 | 2-(2H-benzotriazol-2-yl)-4-methyl- | 155633-54-8 |
| | 6-[2-methyl-3[1,3,3,3-tetramethyl-1- | |
| | [(trimethylsilyl)oxy]disiloxanyl]propyl]phenol | |
| 38 | 1,1-[(2,2′-dimethylpropoxy)carbonyl]- | 363602-15-7 |
| | 4,4-diphenyl-1,3-butadiene | |

In addition to the two abovementioned groups of primary sunscreen substances, secondary sunscreen agents of the antioxidant type, which interrupt the photochemical reaction chain triggered when UV radiation penetrates into the skin, can also be employed. Typical examples of these are superoxide dismutase, catalase, tocopherols (vitamin E) and ascorbic acid (vitamin C).

A further group are anti-irritants, which have an anti-inflammatory action on skin damaged by UV light. Such substances are, for example, bisabolol, phytol and phytantriol.

The invention also provides the use of the polymers according to the invention or prepared by the process according to the invention which are molecularly imprinted with active compound in dermocosmetic formulations in combination with inorganic pigments which keep out UV rays. Pigments based on metal oxides and/or other metal compounds which are sparingly soluble or insoluble in water chosen from the group consisting of oxides of zinc (ZnO), titanium (TiO₂), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals and blends of such oxides are preferably present.

In this context, the inorganic pigments can be in a coated form, i.e. they are treated on the surface. This surface treatment can comprise, for example, providing the pigments with a thin hydrophobic layer in a manner known per se, as described in DE-A-33 14 742.

Suitable repellent active compounds are compounds which are capable of keeping off or driving away certain animals, in particular insects, from humans. These include e.g. 2-ethyl-1,3-hexanediol, N,N-diethyl-m-toluamide etc. Suitable substances having a hyperemizing action which stimulate circulation of blood to the skin are e.g. essential oils, such as dwarf pine extract, lavender extract, rosemary extract, juniper berry extract, horse chestnut extract, birch leaf extract, hayseed extract, ethyl acetate, camphor, menthol, peppermint oil, rosemary extract, eucalyptus oil etc. Suitable keratolytically and keratoplastically acting substances are e.g. salicylic acid, calcium thioglycolate, thioglycolic acid and its salts, sulfur etc. Suitable antidandruff active compounds are e.g. sulfur, sulfur-polyethylene glycol sorbitan monooleate, sulfur-ricinoleyl polyethoxylate, zinc pyrithione, aluminum pyrithione etc. Suitable antiphlogistics, which counteract irritation of the skin, are e.g. allantoin, bisabolol, dragosantol, chamomile extract, panthenol etc.

The invention also provides the use of the polymers according to the invention or prepared by the process according to the invention which are molecularly imprinted with active compound in combination with at least one cosmetically or pharmaceutically acceptable polymer.

Suitable polymers are e.g. cationic polymers with the INCI name polyquaternium, e.g. copolymers of vinylpyrrolidone/N-vinylimidazolium salts (Luviquat FC, Luviquat HM, Luviquat MS, Luviquat&commat, Care), copolymers of N-vinylpyrrolidone/dimethylaminoethyl methacrylate, quaternized with diethyl sulfate (Luviquat PQ 11), copolymers of N-vinylcaprolactam/N-vinylpyrrolidone/N-vinyl-imidazolium salts (Luviquat E Hold), cationic cellulose derivatives (polyquaternium-4 and -10), acrylamido copolymers (polyquaternium-7) and chitosan.

Suitable cationic (quaternized) polymers are also Merquat (polymer based on dimethyldiallylammonium chloride), Gafquat (quaternary polymers which are formed by reaction of polyvinylpyrrolidone with quaternary ammonium compounds), Polymer JR (hydroxyethylcellulose with cationic groups) and plant-based cationic polymers, e.g. guar polymers, such as the Jaguar brands from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives and polyaspartic acid salts and derivatives. These include, for example, Luviflex 0 Swing (partly saponified copolymer of polyvinyl acetate and polyethylene glycol, BASF Aktiengesellschaft).

Suitable polymers are also nonionic, water-soluble or water-dispersible polymers or oligomers, such as polyvinylcaprolactam, e.g. Luviskol 0 Plus (BASF), or polyvinylpyrrolidone and copolymers thereof, in particular with vinyl esters, such as vinyl acetate, e.g. Luviskol 0 VA 37 (BASF), and polyamides, e.g. based on itaconic acid and aliphatic diamines, such as are described e.g. in DE-A-43 33 238.

Suitable polymers are also amphoteric or zwitterionic polymers, such as the octylacrylamide/methyl methacrylate/tert-butylaminoethyl methacrylate-hydroxypropyl methacrylate copolymers obtainable under the name Amphomer (National Starch) and zwitterionic polymers such as are disclosed, for example, in the German Patent Applications DE 39 29 973, DE 21 50 557, DE 28 17 369 and DE 37 08 451. Acrylamidopropyltrimethylammonium chloride/acrylic acid or methacrylic acid copolymers and alkali metal and ammonium salts thereof are preferred zwitterionic polymers. Zwitterionic polymers which are furthermore suitable are methacroylethylbetaine/methacrylate copolymers which are commercially obtainable under the name Amersette (AMERCHOL), and copolymers of hydroxyethyl methacrylate, methyl methacrylate, N,N-dimethylaminoethyl methacrylate and acrylic acid (Jordapon (D)).

Suitable polymers are also nonionic, siloxane-containing, water-soluble or -dispersible polymers, e.g. polyether-siloxanes, such as Tegopren 0 (Goldschmidt) or Besi&commat (Wacker).

The invention also provides the use of the molecularly imprinted polymers in combination with dermocosmetic active compounds (one or more compounds) advantageously chosen from the group consisting of acetylsalicylic acid, atropine, azulene, hydrocortisone and derivatives thereof, e.g. hydrocortisone 17-valerate, vitamins of the B and D series, in particular vitamin B₁, vitamin B₁₂, vitamin D, vitamin A and derivatives thereof, such as retinyl palmitate, vitamin E or derivatives thereof, such as e.g. tocopheryl acetate, vitamin C and derivatives thereof, such as e.g. ascorbyl glucoside, and also niacinamide, panthenol, bisabolol polydocanol, unsaturated fatty acids, such as e.g. the essential fatty acids (conventionally called vitamin F), in particular γ-linolenic acid, oleic acid, eicosapentaenoic acid, docosahexaenoic acid and derivatives thereof, chloramphenicol, caffeine, prostaglandins, thymol, camphor, squalene, extracts or other products of plant and animal origin, e.g. evening primrose oil, borage oil or carob bean oil, fish oils, cod-liver oil, and also ceramides and ceramide-like compounds, incense extract, green tea extract, water lily extract, liquorice extract, witch hazel, antidandruff active compounds (e.g. selenium disulfide, zinc pyrithione, piroctone, olamine, climbazole, octopirox, polydocanol and combinations thereof) and complexed active compounds, such as e.g. those of γ-oryzanol and calcium salts, such as calcium pantothenate, calcium chloride and calcium acetate. It is also advantageous to choose the active compounds from the group consisting of re-oiling substances, for example purcellin oil, Eucerit® and Neocerit®. The active compound or compounds are furthermore particularly advantageously chosen from the group consisting of NO synthase inhibitors, in particular if the formulations according to the invention are to be used for treatment and prophylaxis of the symptoms of intrinsic and/or extrinsic ageing of the skin and for treatment and prophylaxis of the harmful effects of ultraviolet radiation on the skin and hair. Nitroarginine is the preferred NO synthase inhibitor. The active compound or compounds are furthermore advantageously chosen from the group including catechols and bile acid esters of catechols and aqueous or organic extracts from plants or plant parts which have a content of catechols or bile acid esters of catechols, such as, for example, the leaves of the Theaceae plant family, in particular of the species Camellia sinensis (green tea). Typical constituents thereof (e.g. polyphenols or catechols, caffeine, vitamins, sugars, minerals, amino acids, lipids) are particularly advantageous. Catechols are a group of compounds which are to be interpreted as hydrogenated flavones or anthocyanidines and are derivatives of “catechol” (catechol, 3,3′,4′,5,7-flavanepentaol, 2-(3,4-dihydroxyphenyl)-chromane-3,5,7-triol). Epicatechol ((2R,3R)-3,3′,4′,5,7-flavanepentaol) is also an advantageous active compound in the context of the present invention. Plant extracts having a content of catechol, in particular extracts of green tea, such as e.g. extracts from leaves of the plants of the species Camellia spec., very particularly the tea varieties Camellia sinensis, C. assamica, C. taliensis or C. inawadiensis, and cross-varieties of these with, for example, Camellia japonica, are furthermore advantageous. Preferred active compounds are furthermore polyphenols or catechols from the group consisting of (−)-catechol, (+)-catechol, (−)-catechol gallate, (−)-gallocatechol gallate, (+)-epicatechol, (−)-epicatechol, (−)-epicatechol gallate, (−)-epigallocatechol and (−)-epigallocatechol gallate.

Flavone and its derivatives (often also collectively called “flavones”) are also advantageous active compounds in the context of the present invention. They are characterized by the following basic structure (substitution positions shown):

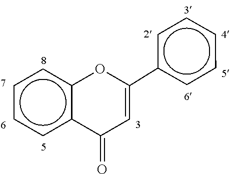

Some of the more important flavones, which can also preferably be employed in formulations according to the invention, are listed in the following Table 8.

TABLE 7
Flavones

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones as a rule occur in the glycosidated form.

According to the invention, the flavonoids are preferably chosen from the group consisting of substances of the general formula

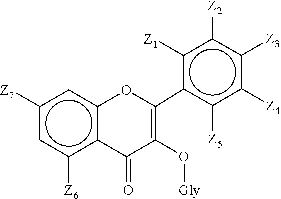

wherein Z₁ to Z₇ are chosen independently of one another from the group consisting of H, OH, alkoxy- and hydroxyalkoxy-, wherein the alkoxy and hydroxyalkoxy groups can be branched and unbranched and can contain 1 to 18 C atoms, and wherein Gly is chosen from the groups consisting of mono- and oligoglycoside radicals.

The active compounds (one or more compounds) can moreover also very advantageously be chosen from the groups consisting of hydrophilic active compounds, in particular from the following group:

α-hydroxy acids, such as lactic acid or salicylic acid, or salts thereof, such as e.g. Na lactate, Ca lactate or TEA lactate, urea, allantoin, serine, sorbitol, glycerol, milk proteins, panthenol or chitosan.

The amount of such active compounds (one or more compounds) in the formulations according to the invention is preferably 0.001 to 30 wt. %, particularly preferably 0.05 to 20 wt. %, in particular 1 to 10 wt. %, based on the total weight of the formulation. The active compounds mentioned and further active compounds which can be used in the formulations according to the invention are mentioned in DE 103 18 526 A1 on pages 12 to 17, to which reference is made in its full scope at this point.

The present invention furthermore relates to the use of the abovementioned formulations for preventing undesirable changes to the skin profile, such as e.g. acne or greasy skin, keratoses, rosaceae or photosensitive, inflammatory, erythematous, allergic or autoimmune-reactive reactions.

The compositions according to the invention are preferably skin protection compositions, skin care compositions, skin cleansing compositions, hair protection compositions, hair care compositions, hair cleansing compositions, hair coloring compositions, mouth washes and mouth rinses, or formulations for decorative cosmetics, which are preferably used in the form of ointments, creams, emulsions, suspensions, lotions, milk, pastes, gels, foams or sprays, depending on the field of use.

In addition to the polymers according to the invention or prepared by the process according to the invention which are molecularly imprinted with active compound, the dermocosmetics according to the invention can comprise all the polymers, pigments, humectants, oils, waxes, enzymes, minerals, vitamins, sunscreen agents, dyestuffs, fragrances, antioxidants, preservatives and/or pharmaceutical active compounds listed above.

The following furthermore applies to the dermocosmetics according to the invention:

The formulation base of compositions according to the invention preferably comprises cosmetically or dermocosmetically/pharmaceutically acceptable auxiliaries. The auxiliaries which are known to be usable in the field of pharmacy, foodstuffs technology and related areas are pharmaceutically acceptable, in particular the auxiliaries listed in relevant pharmacopeias (e.g. DAB, Ph. Eur., BP, NF) and other auxiliaries, the properties of which do not rule out a physiological use.

Suitable auxiliaries can be: lubricants, wetting agents, emulsifying and suspending agents, preservatives, antioxidants, anti-irritants, chelating agents, emulsion stabilizers, film-forming agents, gelling agents, odor masking agents, resins, hydrocolloids, solvents, solubilizing agents, neutralizing agents, permeation accelerators, pigments, quaternary ammonium compounds, re-oiling and super-oiling agents, ointment, cream or oil bases, silicone derivatives, stabilizers, sterilizing agents, propellants, desiccants, opacifying agents, thickeners, waxes, softeners and white oil. A formulation in this respect is based on expert knowledge, such as is contained, for example, in Fiedler, H. P. Lexikon der Hilfsstoffe für Pharmazie, Kosmetik and angrenzende Gebiete [Dictionary of Auxiliaries for Pharmacy, Cosmetics and Related Fields], 4th ed., Aulendorf: ECV-Editio-Kantor-Verlag, 1996.

For preparation of the dermocosmetic compositions according to the invention, the active compounds can be mixed or diluted with a suitable auxiliary (excipient). Excipients can be solid, semi-solid or liquid materials, which can serve as a vehicle, carrier or medium for the active compound. If desired, further auxiliaries are admixed in the manner known to the person skilled in the art. The polymers and dispersions are furthermore suitable as auxiliaries in pharmacy, preferably as or in coating compositions or binders for solid medicament forms. They can also be used in creams and as tablet-coating compositions and tablet binders.

According to a further preferred embodiment, the compositions according to the invention are cosmetic compositions for care and protection of the skin and hair, nail care compositions or formulations for decorative cosmetics.

Suitable skin cosmetic compositions are e.g. face lotions, face masks, deodorants and other cosmetic lotions. Compositions for use in decorative cosmetics include, for example, covering sticks, grease paints, mascara and eye shadow, lipsticks, kajal sticks, eyeliners, rouges, powders and eyebrow pencils.

The molecularly imprinted polymers can moreover be used in nose strips for cleansing pores, in anti-acne compositions, repellents, shaving compositions, after- and pre-shave care compositions, after-sun care compositions, depilatory compositions, hair coloring compositions, intimate care compositions, foot care compositions and in baby care.

The skin care compositions according to the invention are, in particular, W/O or O/W skin creams, day and night creams, eye creams, face creams, antiwrinkle creams, sunscreen creams, moisturizing creams, bleaching creams, self-tanning creams, vitamin creams, skin lotions, care lotions and moisturizing lotions.

In addition to the polymers according to the invention or prepared by the process according to the invention which are molecularly imprinted with active compound, skin cosmetic and dermatological compositions according to the invention can furthermore comprise an active compound which destroys free radicals, as protection against oxidative processes and the associated ageing processes in or damage to skin and/or hair. These active compounds are preferably the substances described in the patent applications WO/0207698 and WO/03059312, to the content of which reference is herewith expressly made, preferably the boron-containing compounds described there, which can reduce peroxides or hydroperoxides to the corresponding alcohols without the formation of free-radical secondary stages. Sterically hindered amines according to the general formula

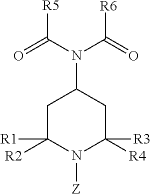

wherein the radical Z has the following meaning: H, C1-C22 alkyl group, preferably C1-C12 alkyl group, such as methyl, ethyl, propyl, isopropyl, butyl, isobutyl, sec-butyl, tert-butyl, pentyl, isopentyl, neopentyl, tert-pentyl, hexyl, heptyl, octyl, nonyl, decyl, undecyl or dodecyl, C1-C22-alkoxy group, preferably C1-C12 alkoxy group, such as alkoxy-methyl, alkoxy-ethyl, alkoxy-propyl, alkoxy-isopropyl, alkoxy-butyl, alkoxy-isobutyl, alkoxy-sec-butyl, alkoxy-tert-butyl, alkoxy-pentyl, alkoxy-isopentyl, alkoxy-neopentyl, alkoxy-tert-pentyl, alkoxy-hexyl, alkoxy-heptyl, alkoxy-octyl, alkoxy-nonyl, alkoxy-decyl, alkoxy-undecyl or alkoxy-dodecyl, C6 to C10 aryl group, such as phenyl and naphthyl, wherein the phenyl radical can be substituted by C1 to C4 alkyl radicals, or C6 to C10 O-aryl group, which can be substituted by a C1-C22 alkyl or C1-C22 alkoxy group, preferably with a C1-C12 alkyl or C1-C12 alkoxy group as described above, and
the radicals R1 to R6 independently of one another have the following meaning: H, OH, O, C1-C22 alkyl group, preferably C1-C12 alkyl group, such as methyl, ethyl, propyl, isopropyl, butyl, isobutyl, sec-butyl, tert-butyl, pentyl, isopentyl, neopentyl, tert-pentyl, hexyl, heptyl, octyl, nonyl, decyl, undecyl or dodecyl, C1-C22 alkoxy group, preferably C1-C12 alkoxy group, such as alkoxy-methyl, alkoxy-ethyl, alkoxy-propyl, alkoxy-isopropyl, alkoxy-butyl, alkoxy-isobutyl, alkoxy-sec-butyl, alkoxy-tert-butyl, alkoxy-pentyl, alkoxy-isopentyl, alkoxy-neopentyl, alkoxy-tert-pentyl, alkoxy-hexyl, alkoxy-heptyl, alkoxy-octyl, alkoxy-nonyl, alkoxy-decyl, alkoxy-undecyl or alkoxy-dodecyl, C6 to C10 aryl group, such as phenyl and naphthyl, wherein the phenyl radical can be substituted by C1 to C4 alkyl radicals, or C6 to C10 O-aryl group, which can be substituted by a C1-C22 alkyl or C1-C22 alkoxy group, preferably by a C1-C12 alkyl or C1-C12 alkoxy group as described above, can furthermore be used for this purpose.

The use of the sterically hindered amines 3-dodecyl-N-(2,2,6,6-tetramethyl-4-piperidinyl)succinimide, 3-dodecyl-N-(1,2,2,6,6-penta-methyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide, 3-octenyl-N-(1,2,2,6,6-pentamethyl-4-piperidinyl)succinimide and/or Uvinul® 5050H in a content of from 0.001 to 1 percent by weight (wt. %), preferably 0.01 to 0.1 wt. %, 0.1 to 1 wt. %, based on the total weight of the composition, is particularly preferred.

In addition to the abovementioned compounds according to the invention and suitable carriers, the skin cosmetic formulations can also comprise further conventional active compounds and auxiliaries in skin cosmetics, as described above. These include, preferably, emulsifiers, preservatives, perfume oils, cosmetic active compounds, such as phytantriol, vitamin A, E and C, retinol, bisabolol, panthenol, sunscreen agents, bleaching agents, coloring agents, tinting agents, tanning agents, collagen, protein hydrolysates, stabilizers, pH regulators, dyestuffs, salts, thickeners, gelling agents, agents which impart consistency, silicones, humectants, re-oiling agents and/or further conventional additives.

Preferred oil and fat components of the skin cosmetic and dermocosmetic compositions are the abovementioned mineral and synthetic oils, such as e.g. paraffins, silicone oils and aliphatic hydrocarbons having more than 8 carbon atoms, animal and plant oils, such as e.g. sunflower oil, coconut oil, avocado oil, olive oil or lanolin, or waxes, fatty acids, fatty acid esters, such as e.g. triglycerides of C₆-C₃₀ fatty acids, wax esters, such as e.g. jojoba oil, fatty alcohols, vaseline, hydrogenated lanolin and acetylated lanolin and mixtures thereof.

To establish particular properties, such as e.g. improvement in the touch sensation, spreading properties, water resistance and/or binding of active compounds and auxiliaries, such as pigments, the skin cosmetic and dermocosmetic formulations can additionally also comprise conditioning substances based on silicone compounds.

Suitable silicone compounds are, for example, polyalkylsiloxanes, polyarylsiloxanes, polyarylalkylsiloxanes, polyether-siloxanes or silicone resins.

The cosmetic or dermocosmetic formulations are prepared by conventional processes known to the person skilled in the art.

The cosmetic and dermocosmetic compositions are preferably in the form of emulsions, in particular water-in-oil (W/O) or oil-in-water (O/W) emulsions.

However, it is also possible to choose other types of formulation, for example gels, oils, oleogels, multiple emulsions, for example in the form of W/O/W or O/W/O emulsions, anhydrous ointments or ointment bases etc. Emulsifier-free formulations, such as hydrodispersions, hydrogels or a Pickering emulsion, are also advantageous embodiments.

The preparation of emulsions is carried out by known methods. In addition to at least one molecularly imprinted polymer, the emulsions as a rule contain conventional constituents, such as fatty alcohols, fatty acid esters, and in particular fatty acid triglycerides, fatty acids, lanolin and derivatives thereof, natural or synthetic oils or waxes and emulsifiers in the presence of water. The choice of additives specific for the type of emulsion and the preparation of suitable emulsions is described, for example, in Schrader, Grundlagen and Rezepturen der Kosmetika [Principles and Recipes of Cosmetics], Hüthig Buch Verlag, Heidelberg, 2nd edition, 1989, part 3, or Umbach, Kosmetik: Entwicklung, Herstellung and Anwendung kosmetischer Mittel [Cosmetics: Development, Manufacture and Use of Cosmetic Agents], 2nd expanded edition, 1995, Georg Thieme Verlag, ISBN 3 13 712602 9, pages 122 et seq., to which reference is herewith expressly made.

A suitable emulsion as a W/O emulsion, e.g. for a skin cream etc., in general contains an aqueous phase, which is emulsified in an oily or fatty phase by means of a suitable emulsifier system. A polyelectrolyte complex can be employed to provide the aqueous phase.

Preferred fat components which the fat phase of the emulsions can contain are: hydrocarbon oils, such as paraffin oil, purcellin oil, perhydrosqualene and solutions of microcrystalline waxes in these oils; animal or plant oils, such as sweet almond oil, avocado oil, calophylum oil, lanolin and derivatives thereof, castor oil, sesame oil, olive oil, jojoba oil, karite oil, hoplostethus oil, mineral oils, the start of distillation of which under atmospheric pressure is at approx. 250° C. and the distillation end point of which is at 410° C., such as e.g. vaseline oil, and esters of saturated or unsaturated fatty acids, such as alkyl myristates, e.g. i-propyl, butyl or cetyl myristate, hexadecyl stearate, ethyl or i-propyl palmitate, octanoic or decanoic acid triglycerides and cetyl ricinoleate.

The fatty phase can also contain silicone oils which are soluble in other oils, such as dimethylpolysiloxane, methylphenylpolysiloxane and silicone-glycol copolymer, fatty acids and fatty alcohols.

In addition to the compounds according to the invention described above, the skin care compositions can also comprise waxes, such as e.g. carnauba wax, candelilla wax, beeswax, microcrystalline wax, ozocerite wax and Ca, Mg and Al oleates, myristates, linoleates and stearates.

An emulsion according to the invention can also be in the form of an O/W emulsion. Such an emulsion conventionally contains an oily phase, emulsifiers which stabilize the oily phase in the aqueous phase and an aqueous phase, which is conventionally in thickened form. Possible emulsifiers are preferably O/W emulsifiers, such as polyglycerol esters, sorbitan esters or partly esterified glycerides.

According to a further preferred embodiment, the compositions according to the invention are a sunscreen composition, a shower gel, a shampoo formulation or a bath preparation, sunscreen preparations being particularly preferred.

Such formulations comprise at least one polymer according to the invention or prepared by the process according to the invention which is molecularly imprinted with active compound and conventionally anionic surfactants as the base surfactants and amphoteric and/or nonionic surfactants as co-surfactants. Further suitable active compounds and/or auxiliaries are in general chosen from lipids, perfume oils, dyestuffs, organic acids, preservatives and antioxidants, and thickeners/gelling agents, skin conditioning agents and humectants.

These formulations preferably comprise 2 to 50 wt. %, preferably 5 to 40 wt. %, particularly preferably 8 to 30 wt. % of surfactants, based on the total weight of the formulation.

All the anionic, neutral, amphoteric or cationic surfactants conventionally employed in body cleansing compositions can be used in the washing, shower and bath preparations.

Suitable anionic surfactants are, for example, alkyl sulfates, alkyl ether-sulfates, alkylsulfonates, alkylarylsulfonates, alkyl succinates, alkyl sulfosuccinates, N-alkoylsarcosinates, acyl taurates, acyl isothionates, alkyl phosphates, alkyl ether-phosphates, alkyl ether-carboxylates and alpha-olefinsulfonates, in particular the alkali metal and alkaline earth metal salts, e.g. sodium, potassium, magnesium and calcium, as well as ammonium and triethanolamine salts. The alkyl ether-sulfates, alkyl ether-phosphates and alkyl ether-carboxylates can contain between 1 to 10 ethylene oxide or propylene oxide units, preferably 1 to 3 ethylene oxide units, in the molecule.

These include e.g. sodium lauryl-sulfate, ammonium lauryl-sulfate, sodium lauryl ether-sulfate, ammonium lauryl ether-sulfate, sodium lauryl-sarcosinate, sodium oleyl-succinate, ammonium lauryl-sulfosuccinate, sodium dodecylbenzenesulfonate and triethanolamine dodecylbenzenesulfonate.

Suitable amphoteric surfactants are e.g. alkylbetaines, alkylamidopropylbetaines, alkylsulfobetaines, alkyl glycinates, alkyl carboxyglycinates, alkyl amphoacetates or -propionates and alkyl amphodiacetates or -dipropionates.

For example, cocodimethylsulfopropylbetaine, laurylbetaine, cocamidopropylbetaine or sodium cocamphopropionate can be employed.

Suitable nonionic surfactants are, for example, the reaction products of aliphatic alcohols or alkylphenols having 6 to 20 C atoms in the alkyl chain, which can be linear or branched, with ethylene oxide and/or propylene oxide. The amount of alkylene oxide is approx. 6 to 60 mol per mol of alcohol. Alkylamine oxides, mono- or dialkylalkanolamides, fatty acid esters of polyethylene glycols, ethoxylated fatty acid amides, alkyl polyglycosides or sorbitan ether-esters are furthermore suitable.

The washing, shower and bath preparations can moreover comprise conventional cationic surfactants, such as e.g. quaternary ammonium compounds, for example cetyltrimethylammonium chloride.

The shower gel/shampoo formulations can furthermore comprise thickeners, such as e.g. sodium chloride, PEG-55, propylene glycol oleate, PEG-120 methylglucose dioleate and others, as well as preservatives, further active compounds and auxiliaries and water.

### Hair Treatment Compositions

According to a further preferred embodiment, the dermocosmetics according to the invention are hair treatment compositions.

The hair treatment compositions according to the invention are preferably in the form of a foam setting composition, hair mousse, hair gel, shampoo, hair spray, hair foam, spray liquid, leveling composition for permanent waves, hair coloring and bleaching composition or “hot oil treatment”. Depending on the field of use, the hair cosmetic formulations can be applied as a(n) (aerosol) spray, (aerosol) foam, gel, gel spray, cream, lotion or wax. In this context, hair sprays include both aerosol sprays and pump sprays without a propellant gas. Hair foams include both aerosol foams and pump foams without a propellant gas. Hair sprays and hair foams preferably contain predominantly or exclusively water-soluble or water-dispersible components. If the compounds employed in the hair sprays and hair foams according to the invention are water-dispersible, they can be used in the form of aqueous microdispersions having particle diameters of conventionally 1 to 350 nm, preferably 1 to 250 nm. In this context, the solids contents of these preparation are conventionally in a range of from about 0.5 to 20 wt. %. These microdispersions as a rule require no emulsifiers or surfactants for their stabilization.

Further constituents are to be understood as meaning the conventional additives in cosmetics, for example propellants, defoamers and surface-active compounds, i.e. surfactants, emulsifiers, foam-forming agents and solubilizing agents. The surface-active compounds employed can be anionic, cationic, amphoteric or neutral. Further conventional constituents can furthermore be e.g. preservatives, perfume oils, opacifying agents, active compounds, UV filters, care substances, such as panthenol, collagen, vitamins, protein hydrolysates, alpha- and beta-hydroxycarboxylic acids, stabilizers, pH regulators, dyestuffs, viscosity regulators, gelling agents, salts, humectants, re-oiling agents, complexing agents and further conventional additives.

These furthermore include all styling and conditioning polymers known in cosmetics which can be employed in combination with the molecularly imprinted polymers if quite specific properties are to be established.

Suitable conventional polymers of hair cosmetics are, for example, the abovementioned cationic, anionic, neutral, nonionic and amphoteric polymers, to which reference is made here.

To establish particular properties, the formulations can additionally also comprise conditioning substances based on silicone compounds. Suitable silicone compounds are, for example, polyalkylsiloxanes, polyarylsiloxanes, polyarylalkylsiloxanes, polyether-siloxanes, silicone resins or dimethicone copolyols (CTFA) and amino-functional silicone compounds, such as amodimethicone (CTFA).

Propellants are the propellants conventionally used for hair sprays or aerosol foams. Mixtures of propane/butane, pentane, dimethyl ether, 1,1-difluoroethane (HFC-152 a), carbon dioxide, nitrogen or compressed air are preferred.

Emulsifiers which can be used are all the emulsifiers conventionally employed in hair foams. Suitable emulsifiers can be nonionic, cationic or anionic or amphoteric. Examples of nonionic emulsifiers (INCI nomenclature) are laureths, e.g. laureth-4; ceteths, e.g. ceteth-1, polyethylene glycol cetyl ethers, ceteareths, e.g. ceteareth-25, polyglycol fatty acid glycerides, hydroxylated lecithin, lactyl esters of fatty acids and alkyl polyglycosides.

Examples of cationic emulsifiers are cetyldimethyl-2-hydroxyethylammonium dihydrogen phosphate, cetyltrimonium chloride, cetyltrimonium bromide, cocotrimonium methyl-sulfate and quaternium-1 to x (INCI).

Anionic emulsifiers can be chosen, for example, from the group consisting of alkyl sulfates, alkyl ether-sulfates, alkylsulfonates, alkylarylsulfonates, alkyl succinates, alkyl sulfosuccinates, N-alkoylsarcosinates, acyl taurates, acyl isethionates, alkyl phosphates, alkyl ether-phosphates, alkyl ether-carboxylates and alpha-olefinsulfonates, in particular the alkali metal and alkaline earth metal salts, e.g. sodium, potassium, magnesium and calcium, as well as ammonium and triethanolamine salts. The alkyl ether-sulfates, alkyl ether-phosphates and alkyl ether-carboxylates can contain between 1 to 10 ethylene oxide or propylene oxide units, preferably 1 to 3 ethylene oxide units, in the molecule.

Gelling agents which can be employed are all the conventional gelling agents in cosmetics. These include weakly crosslinked polyacrylic acid, for example carbomer (INCI), cellulose derivatives, e.g. hydroxypropylcellulose, hydroxyethylcellulose and cationically modified celluloses, polysaccharides, e.g. xanthan gum, caprylic/capric triglyceride, sodium acrylate copolymers, polyquaternium-32 (and) paraffinum liquidum (INCI), sodium acrylate copolymers (and) paraffinum liquidum (and) PPG-1 trideceth-6, arylamidopropyltrimonium chloride/acrylamide copolymers, steareth-10 allyl ether, acrylate copolymers, polyquaternium-37 (and) paraffinum liquidum (and) PPG-1 trideceth-6, polyquaternium 37 (and) propylene glycol dicaprate/dicaprylate (and) PPG-1 trideceth-6, polyquaternium-7 and polyquaternium-44.

All the anionic, neutral, amphoteric or cationic surfactants conventionally employed in shampoos can be used in the shampoo formulations.

Suitable anionic surfactants are, for example, alkyl sulfates, alkyl ether-sulfates, alkylsulfonates, alkylarylsulfonates, alkyl succinates, alkyl sulfosuccinates, N-alkoylsarcosinates, acyl taurates, acyl isothionates, alkyl phosphates, alkyl ether-phosphates, alkyl ether-carboxylates and alpha-olefinsulfonates, in particular the alkali metal and alkaline earth metal salts, e.g. sodium, potassium, magnesium and calcium, as well as ammonium and triethanolamine salts. The alkyl ether-sulfates, alkyl ether-phosphates and alkyl ether-carboxylates can contain between 1 to 10 ethylene oxide or propylene oxide units, preferably 1 to 3 ethylene oxide units, in the molecule.

Sodium lauryl-sulfate, ammonium lauryl-sulfate, sodium lauryl ether-sulfate, ammonium lauryl ether-sulfate, sodium lauroyl-sarcosinate, sodium oleyl-succinate, ammonium lauryl-sulfosuccinate, sodium dodecylbenzenesulfonate and triethanolamine dodecylbenzenesulfonate, for example, are suitable.

Suitable amphoteric surfactants are, for example, alkylbetaines, alkylamidopropylbetaines, alkylsulfobetaines, alkyl glycinates, alkyl carboxyglycinates, alkyl amphoacetates or -propionates and alkyl amphodiacetates or -dipropionates.

For example, cocodimethylsulfopropylbetaine, laurylbetaine, cocamidopropylbetaine or sodium cocamphopropionate can be employed.

Suitable nonionic surfactants are, for example, the reaction products of aliphatic alcohols or alkylphenols having 6 to 20 C atoms in the alkyl chain, which can be linear or branched, with ethylene oxide and/or propylene oxide. The amount of alkylene oxide is approx. 6 to 60 mol per mol of alcohol. Alkylamine oxides, mono- or dialkylalkanolamides, fatty acid esters of polyethylene glycols, alkyl polyglycosides or sorbitan ether-esters are furthermore suitable.

The shampoo formulations can moreover comprise conventional cationic surfactants, such as e.g. quaternary ammonium compounds, for example cetyltrimethylammonium chloride.

Conventional conditioning agents can be employed in combination with the molecularly imprinted polymer in the shampoo formulations in order to achieve particular effects.

These include, for example, the abovementioned cationic polymers with the INCI name polyquaternium, in particular copolymers of vinylpyrrolidone/N-vinylimidazolium salts (Luviquat FC, Luviquat&commat, HM, Luviquat MS, Luviquat Care), copolymers of N-vinylpyrrolidone/dimethylaminoethyl methacrylate, quaternized with diethyl sulfate (Luviquat D PQ 11), copolymers of N-vinylcaprolactam/N-vinylpyrrolidone/N-vinylimidazolium salts (Luviquat D Hold), cationic cellulose derivatives (polyquaternium-4 and -10) and acrylamide copolymers (polyquaternium-7). Protein hydrolysates can furthermore be used, as well as conditioning substance based on silicone compounds, for example polyalkylsiloxanes, polyarylsiloxanes, polyarylalkylsiloxanes, polyether-siloxanes or silicone resins. Further suitable silicone compounds are dimethicone copolyols (CTFA) and amino-functional silicone compounds, such as amodimethicone (CTFA). Cationic guar derivatives, such as guar hydroxypropyltrimonium chloride (INCI) can furthermore be used.

According to a further embodiment, this hair cosmetic or skin cosmetic formulation serves for care or protection of the skin or hair and is in the form of an emulsion, a dispersion, a suspension, an aqueous surfactant formulation, a milk, a lotion, a cream, a balsam, an ointment, a gel, granules, a powder, a stick preparation, such as e.g. a lipstick, a foam, an aerosol or a spray. Such formulations are particularly suitable for topical preparations. Possible emulsions are oil-in-water and water-in-oil emulsions or microemulsions.

As a rule, the hair cosmetic or skin cosmetic formulation is used for application to the skin (topically) or hair. In this context, topical formulations are to be understood as meaning those formulations which are suitable for applying the active compounds to the skin in a fine distribution and preferably in a form which can be absorbed through the skin. Aqueous and aqueous-alcoholic solutions, sprays, foams, foam aerosols, ointments, aqueous gels, emulsions of the O/W or W/O type, microemulsions or cosmetic stick preparations e.g. are suitable for this.

According to a preferred embodiment of the cosmetic composition according to the invention, the composition comprises a carrier. Water, a gas, a water-based liquid, an oil, a gel, an emulsion or microemulsion, a dispersion or a mixture thereof is preferred as the carrier. The carriers mentioned show a good skin tolerability. Aqueous gels, emulsions or microemulsions are particularly advantageous for topical formulations.

Nonionic surfactants, zwitterionic surfactants, ampholytic surfactants or anionic emulsifiers can be used as emulsifiers. The composition according to the invention can comprise the emulsifiers in amounts of from 0.1 to 10, preferably 1 to 5 wt. %, based on the composition.

A surfactant from at least one of the following groups can be used, for example, as a nonionic surfactant:

addition products of from 2 to 30 mol of ethylene oxide and/or 0 to 5 mol of propylene oxide on linear fatty alcohols having 8 to 22 C atoms, on fatty acids having 12 to 22 C atoms and on alkylphenols having 8 to 15 C atoms in the alkyl group;
C_{12/18}-fatty acid mono- and diesters of addition products of from 1 to 30 mol of ethylene oxide on glycerol; glycerol mono- and diesters and sorbitan mono- and diesters of saturated and unsaturated fatty acids having 6 to 22 carbon atoms and ethylene oxide addition products thereof; alkyl mono- and oligoglycosides having 8 to 22 carbon atoms in the alkyl radical and ethoxylated analogues thereof; addition products of from 15 to 60 mol of ethylene oxide on castor oil and/or hydrogenated castor oil; and polyol and, in particular, polyglycerol esters, such as e.g. polyglycerol polyricinoleate, polyglycerol poly-12-hydroxystearate or polyglycerol dimerate. Mixtures of compounds from several of these substance classes are likewise suitable;
addition products of from 2 to 15 mol of ethylene oxide on castor oil and/or hydrogenated castor oil;
partial esters based on linear, branched, unsaturated or saturated C_{6/22}-fatty acids, ricinoleic acid and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose); and mono- di- and trialkyl phosphates and mono-, di- and/or tri-PEG-alkyl phosphates and salts thereof;
wool wax alcohols;
polysiloxane-polyalkyl polyether copolymers and corresponding derivatives;
mixed esters of pentaerythritol, fatty acids, citric acid and fatty alcohol according to DE PS 1165574 and/or mixed esters of fatty acids having 6 to 22 carbon atoms, methylglucose and polyols, preferably glycerol or polyglycerol and polyalkylene glycols.

Zwitterionic surfactants can furthermore be used as emulsifiers. Those surface-active compounds which carry at least one quaternary ammonium group and at least one carboxylate or one sulfonate group in the molecule are called zwitter-ionic surfactants. Particularly suitable zwitterionic surfactants are the so-called betaines, such as the N-alkyl-N,N-dimethylammonium glycinates, for example coco-alkyldimethylammonium glycinate, N-acylaminopropyl-N,N-dimethylammonium glycinates, for example coco-acylaminopropyldimethylammonium glycinate, and 2-alkyl-3-carboxymethyl-3-hydroxyethylimidazolines having in each case 8 to 18 C atoms in the alkyl or acyl group, and coco-acylaminoethylhydroxyethylcarboxymethyl glycinate. The fatty acid amide derivative known under the CTFA name Cocamidopropyl Betaine is particularly preferred.

Ampholytic surfactants are likewise suitable emulsifiers. Ampholytic surfactants are understood as meaning those surface-active compounds which contain, apart from a C_{8,18}-alkyl or -acyl group, at least one free amino group and at least one —COON or —SO₃H group in the molecule and are capable of formation of inner salts. Examples of suitable ampholytic surfactants are N-alkylglycines, N-alkylpropionic acids, N-alkylaminobutyric acids, N-alkyliminodipropionic acids, N-hydroxyethyl-N-alkylamido-propylglycines, N-alkyltaurines, N-alkylsarcosines, 2-alkylaminopropionic acids and alkylaminoacetic acids having in each case about 8 to 18 C atoms in the alkyl group.

Particularly preferred ampholytic surfactants are N-coco-alkylaminopropionate, coco-acylaminoethylaminopropionate and C_{12/18}-acrylsarcosine. In addition to the ampholytic emulsifiers, quaternary emulsifiers are also possible, those of the ester-quat type, preferably methyl-quaternized di-fatty acid triethanolamine ester salts, being particularly preferred. Alkyl ether-sulfates, monoglyceride sulfates, fatty acid sulfates, sulfosuccinates and/or ether-carboxylic acids can moreover be employed as anionic emulsifiers.

Possible oil substances are Guerbet alcohols based on fatty alcohols having 6 to 18, preferably 8 to 10 carbon atoms, esters of linear C₆-C₂₂ fatty acids with linear C₆-C₂₂-fatty alcohols, esters of branched C₆-C₁₃-carboxylic acids with linear C₆-C₂₂-fatty alcohols, esters of linear C₆-C₂₂-fatty acids with branched alcohols, in particular 2-ethylhexanol, esters of linear and/or branched fatty acids with polyhydric alcohols (such as e.g. propylene glycol, dimer diol or trimer triol) and/or Guerbet alcohols, triglycerides based on C₆-C₁₀-fatty acids, liquid mono-/di-, triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-carboxylic acids with linear or branched alcohols having 1 to 22 carbon atoms or polyols having 2 to 10 carbon atoms and 2 to 6 hydroxyl groups, plant oils, branched primary alcohols, substituted cyclohexanes, linear C₆-C₂₂-fatty alcohol carbonates, Guerbet carbonates, esters of benzoic acid with linear and/or branched C₆-C₂₂-alcohols (e.g. Finsolv® TN), dialkyl ethers, ring-opening products of epoxidized fatty acid esters with polyols, silicone oils and/or aliphatic or naphthenic hydrocarbons. Silicone compounds can furthermore also be employed as oil substances, for example dimethylpolysiloxanes, methylphenylpolysiloxanes, cyclic silicones and amino-, fatty acid-, alcohol-, polyether-, epoxy-, fluorine-, alkyl- and/or glycoside-modified silicone compounds, which can be either liquid or resinous at room temperature. The compositions according to the invention can comprise the oil substances in amounts of from 1 to 90, preferably 5 to 80, and in particular 10 to 50 wt. %, based on the composition.

The list of constituents mentioned is not of course intended to be regarded as conclusive or limiting. The constituents can be used individually or in any desired combinations with one another.

### EXAMPLES

### Example 1

### Synthesis of Molecularly Imprinted Polymers with α-tocopherol as the Template Molecule

A 2 liter HWS reactor was equipped with a condenser, a stirrer motor, an anchor stirrer, a nitrogen inlet tube with a glass frit, a Julabo LC 3 laboratory regulator with 2-PT-100 heat sensors, an oil bath with an immersion heater and magnetic stirrer, and 2 HPLC pumps (Bischoff) each having a pump head (0-1 ml/min) for metering the initiator and monomers. Before the start of the experiment, the apparatus was flushed with nitrogen. During the entire experiment, nitrogen was passed through the solution with a volume flow of approx. 101/h. 800 ml of the solvent acetonitrile (AcN) were initially introduced into the reaction vessel, and 17.25 g α-tocopherol (template) were dissolved in this.

An additional solution (solution 1) of 6.12 g methacrylic acid and 73.51 g trimethylolpropane trimethacrylate (TRIM) in 250 ml acetonitrile was prepared. 1 ml was taken from solution 1 for later HPLC analysis. Half of the remaining solution 1 was added to the initial mixture in the reactor and mixed with this, while stirring (100 min⁻¹). A sample of 1 ml was then taken from the reactor for HPLC analysis.

A quarter of the amount of initiator, i.e. 0.532 g, was added to the reactor, and after dissolving of the initiator, which was accelerated by vigorous stirring, a sample of 1 ml was taken for later HPLC analysis.

The initial mixture in the reactor was heated to 75° C., while stirring, and a sample of 1 ml was again taken.

A further solution (solution 2) was prepared, and in particular by dissolving the remaining ¾ of the initiator, corresponding to 1.594 g, in 250 ml acetonitrile.

The second half of solution 1 and solution 2 were metered into the reactor by means of the two HPLC pumps over a period of 18 h. The metering rates were for solution 1: 0.153 ml/min, and for solution 2: 0.232 ml/min.

The subsequent after-reaction time was 6 hours, and the total reaction time was therefore 24 h. After each complete hour, a sample of 1 ml was taken from the reaction mixture and, after filtration, subjected to an HPLC analysis.

After the end of the polymerization, the polymer suspension was removed from the reactor and filtered, using a suction filter. The filter-cake was washed three times with 100 ml acetonitrile each time and dried at 50° C. in vacuo.

Example b) A 2 liter HWS reactor was equipped with a condenser, a stirrer motor, an anchor stirrer, a nitrogen inlet tube with a glass frit, a Julabo LC 3 laboratory regulator with 2-PT-100 heat sensors and an oil bath with an immersion heater and magnetic stirrer. Before the start of the experiment, the apparatus was flushed with nitrogen. During the entire experiment, nitrogen was passed through the solution with a volume flow of approx. 10 l/h. 1,000 ml of the solvent acetonitrile (AcN) were initially introduced into the reaction vessel, and 17.25 g α-tocopherol (template), 6.12 g methacrylic acid and 76.32 g trimethylolpropane trimethacrylate were dissolved in this. The mixture was heated to 65° C., while stirring (100 min⁻¹), and a sample was taken for HPLC analysis. 0.564 g 2,2′-azobis(2-methylbutyronitrile) (initiator) was finally dissolved in 5 ml acetonitrile. This solution was injected slowly into the contents of the reactor. After each complete hour, a sample of 10 ml was taken from the reaction mixture and, after filtration, subjected to HPLC analysis. The total reaction time was 5 hours. After the end of the polymerization, the polymer suspension was removed from the reactor and filtered, using a suction filter. The filter-cake was washed three times with 100 ml acetonitrile each time and dried at 50° C. in vacuo.

### Example 2

### Extraction of the Active Compound from the Polymer

A 500 ml round-bottomed flask was equipped with a Soxhlet apparatus, a condenser, a magnetic stirrer and a laboratory regulator (Julabo LC 3 with 2 PT 100) and immersed in an oil-bath. 8 g of polymer were extracted in the Soxhlet apparatus with 400 ml methanol/glacial acetic acid (7:1, v/v) (EXTRACT 1) for 6-8 hours and thereafter for 6 hours with 400 ml methanol (EXTRACT 2). The extracts were collected, the volume thereof was determined, and in each case a 2 ml sample was stored in a refrigerator at 4° C. for determination of the α-tocopherol concentration by means of HPLC.

### Example 3

### Loading of the Polymer with a Cosmetic Active Compound

After drying of the polymer originating from the Soxhlet extraction, 1 g of polymer was mixed with 10 ml of a 0.14 mol/l fipronil solution. (Preparation of the 0.14 mol/l α-tocopherol solution: Dissolve 3 g α-tocopherol in 50 ml acetonitrile). After an action time of 3 hours, the liquid was separated from the polymer by centrifugation (15 minutes at 3,800 rpm) and decanting. The polymer was dried at 50° C. in vacuo.

### Example 4

### Controlled Release of Cosmetic Active Compounds from Molecularly Imprinted Polymers

A Millipore ultrafiltration cell (model 8400) was connected to a 5 liter canister of plastic as the reservoir vessel (content: water). The cell was filled with a dispersion of 100 mg polymer in 100 ml water and the dispersion was stirred with the magnetic stirrer incorporated into the ultrafiltration cell for 15 minutes, until homogenized. The extraction agent (water) was passed into the cell from the reservoir vessel via the feed without increased pressure. The extract discharged from the cell was passed into a collecting vessel on the under-side of the cell. Finally, the volume, the weight and the time of the individual fractions were determined and a sample of 2 ml was taken from each fraction. Several fractions were collected over the time of the experiment. These samples were filtered over a 0.45 μl filter and stored in a refrigerator at 4° C. for the determination of the α-tocopherol concentration by means of HPLC. After the end of the experiment, the dispersion was removed from the ultrafiltration vessel and transferred to a container.

### Example 5

### Controlled Release of Cosmetic Active Compounds from Molecularly Imprinted Polymers

100 mg of the molecularly imprinted polymer are initially introduced into a 250 ml conical flask. 100 ml of the extraction agent (water with an adjusted pH) are then added, the flask is closed and the suspension is stirred for 6 hours at room temperature. Sampling: every 30 minutes; sample volume: in each case 1 ml. Finally, the product is filtered off with suction and a 1 ml sample is taken from the filtrate for HPLC. 13 samples are collected in this way over the time of the experiment (including the sample at time t=0). These samples are filtered over a 0.45 μl filter, transferred into injection bottles and employed for the tocopherol determination by means of HPLC.

### Examples of Dermocosmetic Formulations

Dermocosmetic formulations according to the invention comprising the molecularly imprinted polymer with α-tocopherol as the template molecule, prepared according to Example 1, are described in the following. The said molecularly imprinted polymer with α-tocopherol as the template molecule is called MIP in the following examples. The molecularly imprinted polymer with α-tocopherol as the template molecule is mentioned in the following examples representatively for all the other molecularly imprinted polymers with active compound which are described. It goes without saying for the person skilled in the art that all other active compounds mentioned can be prepared according to Example 1 and used in the formulations mentioned below.

### Example 8

### Use of the MIP in a Face Cleansing Lotion—O/W Type

### AC 1%:

| | % | Constituent (INCl) |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | Aqueous solution with approx. 5% MIP |
| | 60.7 | Aqua dem. |

### AC 5%:

| | % | Constituent (INCl) |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | Aqueous solution with approx. 5 % MIP |
| | 56.7 | Aqua dem. |

Preparation: Dissolve phase A. Stir phase B into phase A, incorporate phase C into the combined phases A and B. Dissolve phase D, stir into the combined phases A, B and C and homogenize. After-stir for 15 min.

### Example 9

### Use of the MIP in a Daily Care Body Spray

### AC 1%:

| | % | Constituent (INCl) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 1.0 | Aqueous solution with approx. 5% MIP |
| | 59.2 | Alcohol |

### AC 5%:

| | % | Constituent (INCl) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 5.0 | Aqueous solution with approx. 5% MIP |
| | 55.2 | Alcohol |

Preparation: Weigh the components of phase A and dissolve to give a clear solution.

### Example 17

### Use of the MIP in a W/O Emulsion with Bisabolol

### AC 1%:

| | % | Constituent (INCl) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 55.6 | Aqua dem. |
| C | 1.0 | Aqueous solution with approx. 5% MIP |
| | 0.5 | Tocopheryl Acetate |
| | 0.6 | Bisabolol |

### AC 5%:

| | % | Constituent (INCl) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 51.6 | Aqua dem. |
| C | 5.0 | Aqueous solution with approx. 5% MIP |
| | 0.5 | Tocopheryl Acetate |

Preparation: Heat phases A and B to approx. 85° C. separately from one another. Stir phase B into phase A and homogenize. While stirring, cool to approx. 40° C., add phase C and homogenize again briefly. While stirring, cool to room temperature.

### Example 24

### Styling Foam

### AC 1%

| | % | Constituent (INCl) |
|---|---|---|
| A | 2.00 | Cocotrimonium Methosulfate |
| | q.s. | Perfume oil |
| B | 72.32 | Aqua dem. |
| | 2.00 | VP/Acrylates/Lauryl Methacrylate Copolymer |
| | 0.53 | AMP |
| | 1.00 | Aqueous solution with approx. 5% MIP |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

### AC 5%

| | % | Constituent (INCl) |
|---|---|---|
| A | 2.00 | Cocotrimonium Methosulfate |
| | q.s. | Perfume oil |
| B | 68.32 | Aqua dem. |
| | 2.00 | VP/Acrylates/Lauryl Methacrylate Copolymer |
| | 0.53 | AMP |
| | 5.00 | Aqueous solution with approx. 5% MIP |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Dissolve phase C in the mixture of A and B and then adjust the pH to 6-7. Transfer to containers with phase D.

### Example 36

### Liquid Make-Up—O/W Type

### AC 1%

| | % | Constituent (INCl) |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 61.9 | Aqua dem. |
| C | 0.1 | Bisabolol |
| | 1.0 | Aqueous solution with approx. 5 % MIP |
| | q.s. | Perfume oil |
| D | 5.7 | C. I. 77 891. Titanium Dioxide |
| | 1.1 | Iron Oxides |

### AC 5%

| | % | Constituent (INCl) |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 57.9 | Aqua dem. |
| C | 0.1 | Bisabolol |
| | 5.0 | Aqueous solution with approx. 5 % MIP |
| | q.s. | Perfume oil |
| D | 5.7 | C. I. 77 891. Titanium Dioxide |
| | 1.1 | Iron Oxides |

Preparation: Heat phases A and B to approx. 80° C. separately. Stir phase B into phase A and homogenize. While stirring, cool to approx. 40° C., add phases C and D and homogenize thoroughly again. While stirring, allow to cool to room temperature.

### Example 37

Dermocosmetic formulations according to the invention comprising the molecularly imprinted polymer with α-tocopherol as the template molecule, prepared according to Example 1, are described in the following. The said molecularly imprinted polymer with α-tocopherol as the template molecule is called MIP in the following examples. The molecularly imprinted polymer with α-tocopherol as the template molecule is mentioned in the following examples representatively for all the other molecularly imprinted polymers with active compound which are described. It goes without saying for the person skilled in the art that all other active compounds mentioned can be prepared according to Example 1 and used in the formulations mentioned below.

The molecularly imprinted polymer with active compound mentioned is employed as a solid. The following data are parts by weight.

Clear shampoo

| Constituents (INCl) | 1 | 2 | 3 | 4 | 5 |
|---|---|---|---|---|---|
| Sodium Laureth Sulfate | 13.00 | 15.00 | 10.50 | 12.50 | 10.00 |
| Codamidopropyl Betaine | 7.50 | 7.00 | 5.00 | 5.50 | 10.00 |
| PEG-7 Glyceryl Cocoate | 2.00 | 2.50 | 3.50 | 5.00 | 2.30 |
| Perfume oil | 0.10 | 0.10 | 0.10 | 0.10 | 0.10 |
| MIP | 1.0 | 5.0 | 0.1 | 0.5 | 10.0 |
| D-Panthenol USP | 1.00 | 1.50 | 1.80 | 1.70 | 1.40 |
| Preservative | 0.10 | 0.10 | 0.10 | 0.10 | 0.10 |
| Citric Acid | 0.10 | 0.10 | 0.10 | 0.10 | 0.10 |
| Luviquat ® Ultra Care | 1.50 | 1.00 | 1.50 | 1.20 | 1.10 |
| Sodium Chloride | 1.50 | 1.40 | 1.40 | 1.30 | 1.50 |
| Aqua dem. | to 100 | to 100 | to 100 | to 100 | to 100 |

Shampoo

| Constituents (INCl) | 1 | 2 | 3 | 4 | 5 |
|---|---|---|---|---|---|
| Sodium Laureth Sulfate | 35.00 | 40.00 | 30.00 | 45.00 | 27.00 |
| Decyl Glucoside | 5.00 | 5.50 | 4.90 | 3.50 | 7.00 |
| Cocamidopropyl Betaine | 10.00 | 5.00 | 12.50 | 7.50 | 15.00 |
| Perfume oil | 0.10 | 0.10 | 0.10 | 0.10 | 0.10 |
| MIP | 1.0 | 5.0 | 0.1 | 0.5 | 10.0 |
| D-Panthenol USP | 0.50 | 1.00 | 0.80 | 1.50 | 0.50 |
| Preservative | 0.10 | 0.10 | 0.10 | 0.10 | 0.10 |
| Citric Acid | 0.10 | 0.10 | 0.10 | 0.10 | 0.10 |
| Laureth-3 | 0.50 | 2.00 | 0.50 | 0.50 | 2.00 |
| Sodium Chloride | 1.50 | 1.50 | 1.50 | 1.50 | 1.50 |
| Aqua dem. | to 100 | to 100 | to 100 | to 100 | to 100 |

Clear conditioner
shampoo

| Constituents (INCl) | 1 | 2 | 3 | 4 | 5 |
|---|---|---|---|---|---|
| ® Disodium | 10.00 | 15.00 | 20.00 | 12.00 | 17.00 |
| Cocoamphodiacetate | | | | | |
| ® Decyl Glucoside | 5.00 | 6.00 | 7.00 | 8.00 | 4.00 |
| ® Cocamidopropyl | 15.00 | 12.00 | 10.00 | 18.00 | 20.00 |
| Betaine | | | | | |
| Luviquat ® FC 550 | 0.30 | 0.20 | 0.20 | 0.20 | 0.30 |
| Perfume oil | 0.10 | 0.10 | 0.10 | 0.10 | 0.10 |
| MIP | 20.0 | 5.0 | 1.0 | 0.5 | 10.0 |
| Cremophore ® PS 20 | 5.00 | 1.00 | 1.00 | 7.00 | 5.00 |
| Preservative | 0.10 | 0.10 | 0.10 | 0.10 | 0.10 |
| ® Laureth-3 | 2.00 | 1.00 | 0.50 | 2.00 | 2.00 |
| Citric Acid | 0.20 | 0.20 | 0.20 | 0.20 | 0.20 |
| PEG-12 Distearate | 3.00 | 2.00 | 2.00 | 3.00 | 2.50 |
| Aqua dem. | to 100 | to 100 | to 100 | to 100 | to 100 |

Foam O/W emulsions

| | Emulsion 1 | | Emulsion 2 | |
|---|---|---|---|---|
| | wt. % | vol. % | wt. % | vol. % |
| Stearic Acid | 5.00 | | 1.00 | |
| Cetyl Alcohol | 5.50 | | | |
| Cetearyl Alcohol | | | 2.00 | |
| PEG-40 Stearate | 8.50 | | | |
| PEG-20 Stearate | | | 1.00 | |
| Caprylic/Capric | 4.00 | | 2.00 | |
| Triglyceride | | | | |
| C12-15 Alkyl Benzoate | 10.00 | | 15.00 | |
| Cyclomethicone | 4.00 | | | |
| Dimethicone | | | 0.50 | |
| MIP | 5.0 | | 10.0 | |
| Ethylhexyl Isostearate | | | 5.00 | |
| Myristyl Myristate | | | 2.00 | |
| Ceresin | 1.50 | | | |
| Glycerin | | | 3.00 | |
| Hydroxypropyl Starch | 1.00 | | 3.50 | |
| Phosphate | | | | |
| BHT | | | 0.02 | |
| Disodium EDTA | 0.50 | | 0.10 | |
| Perfume oil, preservative | q.s. | | q.s. | |
| coloring agent | q.s. | | q.s. | |
| Potassium Hydroxide | q.s. | | q.s. | |
| Aqua dem. | to 100 | | to 100 | |
| | adjust pH to | | adjust pH to | |
| | 6.5-7.5 | | 5.0-6.0 | |
| Emulsion 1 | | 70 | | |
| Emulsion 2 | | | | 35 |
| Nitrogen | | 30 | | |
| Propane/Butane | | | | 65 |

Conditioner shampoo with pearlescence

| | 1 | 2 | 3 |
|---|---|---|---|
| Polyquaternium-10 | 0.50 | 0.50 | 0.40 |
| Sodium Laureth Sulfate | 9.00 | 8.50 | 8.90 |
| Codamidopropyl Betaine | 2.50 | 2.60 | 3.00 |
| Uvinule ® MS 40 | 1.50 | 0.50 | 1.00 |
| MIP | 1.0 | 5.0 | 0.5 |
| Pearlescent solution | 2.00 | 2.50 | |
| Disodium EDTA | 0.10 | 0.15 | 0.05 |
| Preservative, perfume oil, thickener | q.s. | q.s. | q.s. |
| Aqua dem. | to 100 | to 100 | to 100 |

adjust pH to 6.0

Clear conditioner shampoo

| | 1 | 2 | 3 |
|---|---|---|---|
| Polyquaternium-10 | 0.50 | 0.50 | 0.50 |
| Sodium Laureth Sulfate | 9.00 | 8.50 | 9.50 |
| MIP | 5.0 | 0.1 | 3.0 |
| Uvinul M ® 40 | 1.00 | 1.50 | 0.50 |
| Preservative, perfume oil, thickener | q.s. | q.s. | q.s. |
| Aqua dem. | to 100 | to 100 | to 100 |

adjust pH to 6.0

Clear conditioner shampoo
with volume effect

| | 1 | 2 | 3 |
|---|---|---|---|
| Sodium Laureth Sulfate | 10.00 | 10.50 | 11.00 |
| Uvinul ® MC 80 | 2.00 | 1.50 | 2.30 |
| MIP | 10.0 | 0.1 | 0.5 |
| Cocamidopropyl Betaine | 2.50 | 2.60 | 2.20 |
| Disodium EDTA | 0.01 | 0.10 | 0.01 |
| Preservative, perfume oil, thickener | q.s. | q.s. | q.s. |
| Aqua dem. | to 100 | to 100 | to 100 |

adjust pH to 6.0

Gel cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Uvinul ® A Plus ™ | 2.00 | 1.50 | 0.75 | 1.00 |
| UvaSorb ® k2A | | 3.00 | | |
| Ethylhexyl Bis- | | | | |
| lsopentylbenzoxazolylphenyl | | | | |
| Melamine | | | | |
| Uvinul ® MC 80 | 3.00 | | 1.00 | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Uvinul ® T 150 | 4.00 | | 3.00 | 4.00 |
| Octocrylene | | 4.00 | | |
| Diethylhexyl Butamido Triazone | 1.00 | | | 2.00 |
| Phenylbenzimidazole Sulfonic Acid | 0.50 | | 3.00 | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | 3.50 | 4.00 | 7.00 | 9.00 |
| Titanium Dioxide-microfine | 1.00 | | 3.00 | |
| Zinc Oxide- microfine | | | | 0.25 |
| MIP | 0.1 | 0.5 | 1.0 | 0.02 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | to 100 | to 100 | to 100 | to 100 |

adjust pH to 6.0

O/W sunscreen formulation

| | 1 | 2 | 3 | 4 | 5 | 6 | 7 |
|---|---|---|---|---|---|---|---|
| Glyceryl Stearate SE | 0.50 | 1.00 | 3.00 | | | 1.50 | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Sodium Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| MIP | 10.0 | 0.5 | 3.0 | 5.0 | 0.1 | 0.02 | 7.5 |
| Uvinul ® A Plus ™ | 2.00 | 1.50 | 0.75 | 1.00 | 2.10 | 4.50 | 5.00 |
| UvaSorb ® k2A | | | | | | | |
| Ethylhexyl Bis- | | | | | | | |
| Isopentylbenzoxazolylphenyl | | | | | | | |
| Melamine | | | | | | | |
| Ethylhexyl | | | | | 5.00 | 6.00 | 8.00 |
| Methoxycinnamate Uvinul ® | | | | | | | |
| MC 80 | | | | | | | |
| Bis-Ethylhexyloxyphenol | | 1.50 | | 2.00 | 2.50 | | 2.50 |
| Methoxyphenyl Triazine | | | | | | | |
| Butyl | | | 2.00 | | 2.00 | 1.50 | |
| Methoxydibenzoylmethane | | | | | | | |
| Disodium Phenyl | 2.50 | | 0.50 | 2.00 | | 0.30 | |
| Dibenzimidazole | | | | | | | |
| Tetrasulfonate | | | | | | | |
| Ethyhexyl Triazone Uvinul ® | 4.00 | | 3.00 | 4.00 | | 2.00 | |
| T 150 | | | | | | | |
| Octocrylene | | 4.00 | | | | | 7.50 |
| Diethylhexyl Butamido | 1.00 | | | 2.00 | 1.00 | | 1.00 |
| Triazone | | | | | | | |
| Phenylbenzimidazole | 0.50 | | 3.00 | | | | |
| Sulfonic Acid | | | | | | | |
| Methylene Bis-Benzotriazolyl | 2.00 | | 0.50 | 1.50 | 2.50 | | |
| Tetramethylbutylphenol | | | | | | | |
| Ethylhexyl Salicylate | | | 3.00 | | | | 5.00 |
| Drometrizole Trisiloxane | | | 0.50 | | | 1.00 | |
| Terephthalidene Dicamphor | | 1.50 | | 1.00 | 1.00 | | 0.50 |
| Sulfonic Acid | | | | | | | |
| Diethylhexyl 2,6-Naphthalate | 3.50 | | 7.00 | | 6.00 | 9.00 | |
| Titanium Dioxide-microfine | 1.00 | | 3.00 | | 3.50 | | 1.50 |
| Zinc Oxide-microfine | | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglyceride | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | to 100 | to 100 | to 100 | to 100 | to 100 | to 100 | to 100 |

Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Sodium Carbomer | | 0.20 | | 0.30 | |
| Acrylates/C10-30 Alkyl | 0.50 | | 0.40 | 0.10 | 0.50 |
| Acrylate Crosspolymer | | | | | |
| Xanthan Gum | | 0.30 | 0.15 | | |
| MIP | 5.0 | 0.5 | 3.0 | 0.1 | 10.0 |
| Uvinul ® A Plus ™ | 2.00 | 1.50 | 0.75 | 1.00 | 2.10 |
| UvaSorb ® k2A Ethylhexyl Bis- | | 3.50 | | | |
| Isopentylbenzoxazolylphenyl | | | | | |
| Melamine | | | | | |
| Ethylhexyl Methoxycinnamate | | | | | 5.00 |
| Uvinul ® MC 80 | | | | | |
| Bis-Ethylhexyloxyphenol | | 1.50 | | 2.00 | 2.50 |
| Methoxyphenyl Triazine | | | | | |
| Butyl Methoxydibenzoylmethane | | | 2.00 | | 2.00 |
| Disodium Phenyl | 2.50 | | 0.50 | 2.00 | |
| Dibenzimidazole | | | | | |
| Tetrasulfonate | | | | | |
| Ethyhexyl Triazone | 4.00 | | 3.00 | 4.00 | |
| Uvinul ® T 150 | | | | | |
| Octocrylene | | 4.00 | | | |
| Diethylhexyl Butamido Triazone | 1.00 | | | 2.00 | 1.00 |
| Phenylbenzimidazole | 0.50 | | 3.00 | | |
| Sulfonic Acid | | | | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor | | 1.50 | | 1.00 | 1.00 |
| Sulfonic Acid | | | | | |
| Diethylhexyl 2,6-Naphthalate | | | 7.00 | | 9.00 |
| Titanium Dioxide-microfine | 1.00 | | 3.00 | | 3.50 |
| Zinc Oxide-microfine | | | | 0.25 | |
| C12-15 Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| Biosaccaride Gum-1 | | 2.50 | 0.50 | | 2.00 |
| DMDM Hydantoin | | 0.60 | 0.45 | 0.25 | |
| lodopropynyl Butylcarbannate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | to 100 | to 100 | to 100 | to 100 | to 100 |

W/O sunscreen emulsion

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| MIP | 5.0 | 1.0 | 10.0 | 0.5 | 0.1 |
| Uvinul ® A Plus ™ | 2.00 | 1.50 | 0.75 | 1.00 | 2.10 |
| UvaSorb ® k2A | | 2.00 | | | |
| Ethylhexyl Bis- | | | | | |
| lsopentylbenzoxazolylphenyl | | | | | |
| Melamine | | | | | |
| Ethylhexyl Methoxycinnamate | | | | | 5.00 |
| Uvinul ® MC 80 | | | | | |
| Bis-Ethylhexyloxyphenol | | 1.50 | | 2.00 | 2.50 |
| Methoxyphenyl Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone | 4.00 | | 3.00 | 4.00 | |
| Uvinul ® T 150 | | | | | |
| Octocrylene | | 4.00 | | | |
| Diethylhexyl Butamido | 1.00 | | | 2.00 | 1.00 |
| Triazone | | | | | |
| Phenylbenzimidazole | 0.50 | | 3.00 | | |
| Sulfonic Acid | | | | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor | | 1.50 | | 1.00 | 1.00 |
| Sulfonic Acid | | | | | |
| Diethylhexyl 2,6-Naphthalate | | | 7.00 | | 4.00 |
| Titanium Dioxide-microfine | 1.00 | | 3.00 | | 3.50 |
| Zinc Oxide-microfine | | | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol | | | 2.00 | 8.00 | 4.00 |
| Dicaprylate/Dicaprate | | | | | |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii | | | 3.00 | | |
| (Shea Butter) | | | | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate MgSO₄ | 1.00 | 0.50 | | 0.50 | |
| Magnesium Chloride MgCl₂ | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 1.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume oil | 0.20 | | 0.40 | 0.35 | |
| Aqua dem. | to 100 | to 100 | to 100 | to 100 | to 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera | 1.50 | 2.00 | 2.00 | 1.50 |
| (Carnauba) Wax | | | | |
| Cera Alba | 0.50 | 0.50 | 0.50 | 0.40 |
| C16-40 Alkyl Stearate | | 1.50 | 1.50 | 1.50 |
| MIP | 0.5 | 3.0 | 1.0 | 5.0 |
| Uvinul ® A Plus ™ | 2.00 | 1.50 | 0.75 | 9.00 |
| UvaSorb ® k2A | | 2.00 | | 4.00 |
| Ethylhexyl Bis- | | | | |
| lsopentylbenzoxazolylphenyl | | | | |
| Melamine | | | | |
| Ethylhexyl Methoxycinnamate | | 3.00 | | |
| Uvinul ® MC 80 | | | | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl | 2.50 | | 0.50 | 2.00 |
| Dibenzimidazole | | | | |
| Tetrasulfonate | | | | |
| Ethyhexyl Triazone | 4.00 | | 3.00 | 4.00 |
| Uvinul ® T 150 | | | | |
| Octocrylene | | 4.00 | | |
| Diethylhexyl Butamido Triazone | 1.00 | | | 2.00 |
| Phenylbenzimidazole | 0.50 | | 3.00 | |
| Sulfonic Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | | | 7.00 | |
| Titanium Dioxide-microfine | 1.00 | | 3.00 | |
| Zinc Oxide-microfine | | | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume oil. BHT | 0.10 | 0.25 | | 0.35 |
| Ricinus Communis (Castor) Oil | to 100 | to 100 | to 100 | to 100 |

PIT Emulsion

| | 1 | 2 | 3 | 4 | 5 | 6 | 7 | 8 |
|---|---|---|---|---|---|---|---|---|
| Glyceryl | 0.50 | 2.00 | 3.00 | 5.00 | | 0.50 | 4.00 | |
| Monostearate SE | | | | | | | | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone | | | | 0.50 | | 1.00 | | |
| Copolyol | | | | | | | | |
| Polyglyceryl-2- | | | | 0.75 | 0.30 | | | |
| Dioleate | | | | | | | | |
| MIP | 0.1 | 5.0 | 0.01 | 0.5 | 3.0 | 0.25 | 10.0 | 3.0 |
| Uvinul ® A Plus ™ | 2.00 | 1.50 | 0.75 | 1.00 | 2.10 | 4.50 | 5.00 | 2.10 |
| UvaSorb ® k2A | | | 4.00 | | | | 1.50 | |
| Ethylhexyl Bis- | | | | | | | | |
| Isopentylbenzoxazolyl | | | | | | | | |
| phenyl Melamine | | | | | | | | |
| Ethylhexyl | | | | | 5.00 | 6.00 | 8.00 | 5.00 |
| Methoxycinnamate | | | | | | | | |
| Uvinul ® MC 80 | | | | | | | | |
| Bis- | | 1.50 | | 2.00 | 2.50 | | 2.50 | 2.50 |
| Ethylhexyloxyphenol | | | | | | | | |
| Methoxyphenyl | | | | | | | | |
| Triazine | | | | | | | | |
| Butyl | | | 2.00 | | 2.00 | 1.50 | | 2.00 |
| Methoxydibenzoylmethane | | | | | | | | |
| Disodium Phenyl | 2.50 | | 0.50 | 2.00 | | 0.30 | | |
| Dibenzimidazole | | | | | | | | |
| Tetrasulfonate | | | | | | | | |
| Ethyhexyl Triazone | 4.00 | | 3.00 | 4.00 | | 2.00 | | |
| Uvinul ® T 150 | | | | | | | | |
| Octocrylene | | 4.00 | | | | | 7.50 | |
| Diethylhexyl | 1.00 | | | 2.00 | 1.00 | | 1.00 | 1.00 |
| Butamido Triazone | | | | | | | | |
| Phenylbenzimidazole | 0.50 | | 3.00 | | | | | |
| Sulfonic Acid | | | | | | | | |
| Methylene Bis- | 2.00 | | 0.50 | 1.50 | 2.50 | | | 2.50 |
| Benzotriazolyl | | | | | | | | |
| Tetramethylbutylphenol | | | | | | | | |
| Ethylhexyl Salicylate | | | 3.00 | | | | 5.00 | |
| Drometrizole | | | 0.50 | | | 1.00 | | |
| Trisiloxane | | | | | | | | |
| Terephthalylidene | | 1.50 | | 1.00 | 1.00 | | 0.50 | 1.00 |
| Dicamphor Sulfonic | | | | | | | | |
| Acid | | | | | | | | |
| Diethylhexyl 2,6- | | | 7.00 | | 10.00 | 7.50 | | 8.00 |
| Naphthalate | | | | | | | | |
| Titanium Dioxide- | 1.00 | | 3.00 | | 3.50 | | 1.50 | 3.50 |
| microfine | | | | | | | | |
| Zinc Oxide-microfine | | | | 0.25 | | 2.00 | | |
| C12-15 Alkyl | 3.50 | | | 6.35 | | | | 0.10 |
| Benzoate | | | | | | | | |
| Cocoglyceride | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl | | 0.50 | | | 2.00 | | | |
| Galactomannan | | | | | | | | |
| Hydrogenated Coco- | | | | | 3.00 | 4.00 | | |
| Glycerides | | | | | | | | |
| Behenoxy | | | | | | 1.50 | 2.00 | |
| Dimethicone | | | | | | | | |
| VP/Hexadecene | | | | 1.00 | 1.20 | | | |
| Copolymer | | | | | | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum | | 2.00 | | 3.60 | | 2.00 | | |
| Parkii (Shea Butter) | | | | | | | | |
| Iodopropyl | 0.12 | | | | 0.20 | | | |
| Butylcarbamate | | | | | | | | |
| Biosaccaride Gum-1 | | | | 0.10 | | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume oil | 0.20 | | 0.20 | | 0.24 | 0.16 | 0.10 | 0.10 |
| Aqua dem. | to 100 | to 100 | to 100 | to 100 | to 100 | to 100 | to 100 | to 100 |

Gel cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Luvigel ® EM | 1.50 | 2.50 | 2.80 | 3.50 |
| Xanthan Gum | 0.10 | 0.13 | 0.10 | 0.13 |
| Cetearyl Alcohol | 3.00 | 2.50 | 3.00 | 2.50 |
| 012-15 Alkylbenzoate | 4.00 | 4.50 | 4.00 | 4.50 |
| Caprylic/Capric Triglyceride | 3.00 | 3.50 | 3.00 | 3.50 |
| Titanium Dioxide-microfine | 1.00 | | 1.50 | |
| Zinc Oxide-microfine | | 2.00 | | 0.25 |
| MIP | 0.5 | 10.0 | 3.0 | 5.0 |
| Dihydroxyacetone | | | 3.00 | 5.00 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | to 100 | to 100 | to 100 | to 100 |

adjust pH to 6.0

O/W formulations
self-tanning agent

| | 1 | 2 | 3 | 4 | 5 | 6 | 7 |
|---|---|---|---|---|---|---|---|
| Glyceryl Monostearate | 0.50 | 1.00 | 3.00 | | | 1.50 | |
| SE | | | | | | | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| MIP | 0.1 | 0.5 | 0.025 | 5.0 | 3.0 | 10.0 | 1.0 |
| Dihydroxyacetone | | | 3.00 | 5.00 | | 4 | |
| Titanium Dioxide- | 1.00 | | | | 1.50 | | 1.50 |
| microfine | | | | | | | |
| Zinc Oxide-microfine | | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii | | 2.00 | | | | | |
| (Shea Butter) | | | | | | | |
| VP/Hexadecene | 0.20 | | | 0.50 | | 1.00 | |
| Copolymer | | | | | | | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soja (Soybean) | | | | 0.50 | | 1.50 | 1.00 |
| Oil | | | | | | | |
| Ethylhexylglycerin | 0.30 | | | | | | |
| DMDM Hydantoin | | 0.60 | 0.40 | 0.20 | | | |
| Iodopropynyl | | | | 0.18 | 0.20 | | |
| Butylcarbamate | | | | | | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | to 100 | to 100 | to 100 | to 100 | to 100 | to 100 | to 100 |

O/W Make Up

| | 1 | 2 | 3 | 4 | 5 | 6 | 7 |
|---|---|---|---|---|---|---|---|
| Glyceryl Monostearate | 0.50 | 1.00 | 3.00 | | | 1.50 | |
| SE | | | | | | | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| MIP | 3.0 | 5.0 | 2.0 | 0.5 | 1.0 | 5.0 | 10.0 |
| Titanium Dioxide | 10.00 | 12.00 | 9.00 | 8.50 | 11.00 | 9.50 | 10.00 |
| Iron Oxide | 2.00 | 4.00 | 3.00 | 5.00 | 3.40 | 6.00 | 4.40 |
| Zinc Oxide | | 4.00 | | 2.00 | | 3.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii | | 2.00 | | | | | |
| (Shea Butter) | | | | | | | |
| VP/Hexadecene | 0.20 | | | 0.50 | | 1.00 | |
| Copolymer | | | | | | | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Glycine Soja (Soybean) | | | | 0.50 | | 1.50 | 1.00 |
| Oil | | | | | | | |
| Ethylhexylglycerin | 0.30 | | | | | | |
| DMDM Hydantoin | | 0.60 | 0.40 | 0.20 | | | |
| Iodopropynyl | | | | 0.18 | 0.20 | | |
| Butylcarbamate | | | | | | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | to 100 | to 100 | to 100 | to 100 | to 100 | to 100 | to 100 |

Hydrodispersion self-tanning agent

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl | 0.50 | | 0.40 | 0.10 | 0.50 |
| Acrylate Crosspolymer | | | | | |
| Xanthan Gum | | 0.30 | 0.15 | | |
| MIP | 3.0 | 1.0 | 0.5 | 0.1 | 5.0 |
| Dihydroxyacetone | | | 3.00 | 5.00 | |
| Uvinul ® A Plus ™ | 2.00 | 1.50 | 0.75 | 1.00 | 2.10 |
| Titanium Dioxide- | 1.00 | | 1.00 | | 1.00 |
| microfine | | | | | |
| Zinc Oxide-microfine | | 1.90 | | 0.25 | |
| C12-15 Alkyl Benzoate | 2.00 | 2.50 | | | |
| Dicapryl Ether | | 4.00 | | | |
| Butylene Glycol | 4.00 | | 2.00 | 6.00 | |
| Dicaprylate/Dicaprate | | | | | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii | | 2.00 | | 5.00 | |
| (Shea Butter) | | | | | |
| VP/Hexadecene | 0.50 | | | 0.50 | 1.00 |
| Copolymer | | | | | |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja | | | 1.50 | | 1.00 |
| (Soybean) Oil | | | | | |
| Vitamin E Acetate | 0.50 | | 0.25 | | 1.00 |
| Glucosylrutin | 0.60 | | | 0.25 | |
| DMDM Hydantoin | | 0.60 | 0.45 | 0.25 | |
| lodopropynyl | 0.20 | | | | |
| Butylcarbamate | | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | to 100 | to 100 | to 100 | to 100 | to 100 |

Hydrodispersion after-sun

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl | 0.50 | 0.30 | 0.40 | 0.10 | 0.50 |
| Acrylate Crosspolymer | | | | | |
| Xanthan Gum | | 0.30 | 0.15 | | |
| MIP | 0.1 | 5.0 | 0.5 | 3.0 | 1.0 |
| C12-15 Alkyl Benzoate | 2.00 | 2.50 | | | |
| Dicapryl Ether | | 4.00 | | | |
| Butylene Glycol | 4.00 | | 2.00 | 6.00 | |
| Dicaprylate/Dicaprate | | | | | |
| Dicapryl Carbonate | | 2.00 | 6.00 | | |
| Dimethicone | | 0.50 | 1.00 | | |
| Phenyl Trimethicone | 2.00 | | 0.50 | | |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume oil | 0.20 | | 0.05 | 0.40 | |
| Aqua dem. | to 100 | to 100 | to 100 | to 100 | to 100 |

WO Emulsions

| | 1 | 2 | 3 | 4 | 5 |
|---|---|---|---|---|---|
| Cetyl Dimethicone | | 2.50 | | 4.00 | |
| Polyglyceryl-2 | 5.00 | | | | 4.50 |
| Dipolyhydroxystearate | | | | | |
| PEG-30 | | | 5.00 | | |
| Dipolyhydroxystearate | | | | | |
| MIP | 5.0 | 10.0 | 0.1 | 0.5 | 1.0 |
| Uvinul ® A PIus ™ | 2.00 | 1.50 | 0.75 | 1.00 | 2.10 |
| Titanium Dioxide-microfine | 1.00 | | 3.00 | | 3.50 |
| Zinc Oxide- microfine | | 0.90 | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene glycol Dicaprylate/ | | | 2.00 | 8.00 | 4.00 |
| Dicaprate | | | | | |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii | | | 3.00 | | |
| (Shea Butter) | | | | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate | 1.00 | 0.50 | | 0.50 | |
| Magnesium Chloride | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 7.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume Oil | 0.20 | | 0.40 | 0.35 | |
| Aqua dem. | to 100 | to 100 | to 100 | to 100 | to 100 |

Solids-stabilized emulsion
(Pickering emulsions)

| | 1 | 2 | 3 | 4 | 5 |
|---|---|---|---|---|---|
| Mineral Oil | | | 16.00 | 16.00 | |
| Octyldodecanol | 9.00 | 9.00 | 5.00 | | |
| Caprylic/Capric | 9.00 | 9.00 | 6.00 | | |
| Triglyceride | | | | | |
| C12-15 Alkyl Benzoate | | | | 5.00 | 8.00 |
| Butylene Glycol | | | | | 8.00 |
| Dicaprylate/Dicaprate | | | | | |
| Dicaprylyl Ether | 9.00 | | | 4.00 | |
| Dicaprylyl Carbonate | | 9.00 | | | |
| Hydroxyoctacosanyl | 2.00 | 2.00 | 2.20 | 2.50 | 1.50 |
| Hydroxystearate | | | | | |
| Disteardimonium | 1.00 | 0.75 | | 0.50 | 0.25 |
| Hectorite | | | | | |
| Cera microcristallina + | | 0.35 | | | 5.00 |
| paraffinum liquidum | | | | | |
| Hydroxypropyl | | | 0.10 | | 0.05 |
| Methylcellulose | | | | | |
| Dimethicone | | | | | 3.00 |
| MIP | 1.0 | 0.5 | 0.1 | 3.0 | 5.0 |
| Titanium Dioxide + | | 3.00 | | | |
| Alumina + | | | | | |
| Simethicone + Aqua | | | | | |
| Titanium Dioxide + | | 2.00 | 4.00 | 2.00 | 4.00 |
| Trimethoxycaprylylsilane | | | | | |
| Silica Dimethyl Silylate | 2.50 | | | 6.00 | 2.50 |
| Boron Nitride | | | 1.00 | | |
| Starch/Sodium | 2.00 | | | | |
| Metaphosphate | | | | | |
| Polymer | | | | | |
| Tapioca Starch | | 0.50 | | | |
| Sodium Chloride | 5.00 | 7.00 | 8.50 | 3.00 | 4.50 |
| Glycerin | | | | 1.00 | |
| Trisodium EDTA | 1.00 | 1.00 | 1.00 | 1.00 | 1.00 |
| Vitamin E Acetate | 5.00 | 10.00 | 3.00 | 6.00 | 10.00 |
| Ascorbyl Palmitate | 1.00 | 1.00 | | 1.00 | |
| Methylparaben | | 0.60 | | | 0.20 |
| Propylparaben | | | | | 0.20 |
| Phenoxyethanol | | | 0.20 | | |
| Hexamidine | | | 0.40 | 0.50 | 0.40 |
| Diisethionate | | | | | |
| Diazolidinyl Urea | | | | | 0.08 |
| Ethanol | | | 0.23 | 0.20 | |
| Perfume oil | 5.00 | | 3.00 | 4.00 | |
| Aqua dem. | 0.20 | | 0.30 | 0.10 | |
| | to 100 | to 100 | to 100 | to 100 | to 100 |

Oil gel

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl | 10.00 | 6.00 | 8.00 | 7.00 |
| Tetraisostearate | | | | |
| Polyglyceryl-3 Diisostearate | 2.50 | | | |
| Bis-Diglyceryl | 9.00 | 8.00 | 10.00 | 8.00 |
| Polyacyladipate-2 | | | | |
| Myristyl Myristate | 3.50 | 3.00 | 4.00 | 3.00 |
| Quaternium-18 Bentonite | 5.00 | 5.00 | 6.00 | 6.00 |
| Propylene Carbonate | 15.00 | 20.00 | 18.00 | 19.50 |
| MIP | 1.0 | 0.5 | 3.0 | 5.0 |
| Vitamin E Acetate | 0.50 | 1.00 | | |
| Ascorbyl Palmitate | 0.05 | | 0.05 | |
| Buxus Chinensis (Jojoba) Oil | 2.00 | 1.00 | | 1.00 |
| Perfume oil, BHT | 0.10 | 0.25 | | 0.35 |
| Ricinus Communis (Castor) | to 100 | to 100 | to 100 | to 100 |
| Oil | | | | |

### Example 38

Cosmetic sunscreen formulations comprising a combination of at least one inorganic pigment, preferably zinc oxide and/or titanium dioxide, and organic UV-A and UV-B filters and the molecularly imprinted polymer with α-tocopherol as the template molecule, prepared according to Example 1, are described in the following recipes. The said molecularly imprinted polymer with α-tocopherol as the template molecule is called MIP in the following examples. The molecularly imprinted polymer with α-tocopherol as the template molecule is mentioned in the following examples representatively for all the other molecularly imprinted polymers with active compound which are described. It goes without saying for the person skilled in the art that all other active compounds mentioned can be prepared according to Example 1 and used in the formulations mentioned below.

Preferably, the template molecule in the molecularly imprinted polymer is an organic UV filter, which is released to the skin via the release mechanism described.

The formulations mentioned in the following are prepared in the conventional manner known to the person skilled in the art.

The content of molecularly imprinted polymer with active compound is 100%. The molecularly imprinted polymer according to the invention with active compound can be employed either in the pure form or as an aqueous solution. In the case of the aqueous solution, the content of water must be adapted to suit the particular formulation.

| | | |
|---|---|---|
| A | 7.50 Uvinul MC 80 | Ethylhexyl Cinnamate |
| | 2.00 Uvinul M 40 | Benzophenone-3 |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 T-Lite SF | Titanium Dioxide, Alumina Hydrate, |
| | | Dimethicone/Methicone Copolymer |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 66.20 Water dem. | Aqua dem. |
| D | 1.00 Sepigel 305 | Polyacrylamide, C13-14 Isoparaffin. |
| | | Laureth-7 |
| | 1% MIP | |
| | q.s. | Preservative |

| | | |
|---|---|---|
| A | 7.50 Uvinul MC 80 | Ethylhexyl Cinnamate |
| | 2.00 Uvinul M 40 | Benzophenone-3 |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Z-COTE MAX | Zinc Oxide (and) Diphenyl Capryl |
| | | Methicone |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 66.20 Water dem. | Aqua dem. |
| D | 2.00 Simulgel NS | Hydroxyethyl Acrylate/Sodium |
| | | Acryloyldimethyl Taurate Copolymer, |
| | | Squalane, Polysorbate 60 |
| | 1% MIP | |
| | q.s. | Preservative |

| | | |
|---|---|---|
| A | 7.50 Uvinul MC 80 | Ethylhexyl Cinnamate |
| | 2.00 Uvinul A Plus | Diethylamino Hydroxybenzoyl Hexyl |
| | | Benzoate |
| | 0.80 Rylo PG 11 | Polyglyceryl Dimer Soyate |
| | 1.00 Span 60 | Sorbitan Stearate |
| | 0.50 Vitamin E acetate | Tocopheryl Acetate |
| | 3.00 Dracorin 100 SE | Glyceryl Stearate, PEG-100 Stearate |
| | 1.00 Cremophor CO 410 | PEG-40 Hydrogenated Castor Oil |
| B | 3.00 Z-COTE MAX | Zinc Oxide (and) Diphenyl Capryl |
| | | Methicone |
| | 1.00 Cetiol SB 45 | Butyrospermum Parkii (Shea Butter) |
| | 6.50 Finsolv TN | C12-15 Alkyl Benzoate |
| C | 5.00 Butylene glycol | Butylene Glycol |
| | 0.30 Keltrol | Xanthan Gum |
| | 0.10 Edeta BD | Disodium EDTA |
| | 0.10 Allantoin | Allantoin |
| | 66.20 Water dem. | Aqua dem. |
| D | 2.00 Simulgel NS | Hydroxyethyl Acrylate/Sodium |
| | | Acryloyldimethyl Taurate Copolymer, |
| | | Squalane, Polysorbate 60 |
| | 1% | MIP |
| | q.s. | Preservative |

| | | |
|---|---|---|
| A | 1.00 Abil Care 85 | Bis-PEG/PPG-16/16 PEG/PPG- |
| | | 16/16 Dimethicone, Caprylic/Capric |
| | | Triglyceride |
| | 3.00 Cremophor CO 40 | PEG-40 Hydrogenated Castor Oil |
| | 0.30 Cremophor WO 7 | PEG-7 Hydrogenated Castor Oil |
| | 2.00 Parsol 1789 | Butyl Methoxydibenzoylmethane |
| | 2.00 Mexoryl XL | Drometrizole Trisiloxane |
| | 10.00 Witconol APM | PPG-3 Myristyl Ether |
| | 1.00 Uvinul T 150 | Ethylhexyl Triazone |
| | 1.00 Dow Corning 345 Fluid | Cyclopentasiloxane, |
| | | Cyclohexasiloxane |
| | 5.00 Uvinul N 539 T | Octocrylene |
| B | 3.00 Z-COTE MAX | Zinc Oxide (and) Diphenyl Capryl |
| | | Methicone |
| C | 5.00 1,2-Propylene glycol | Propylene Glycol |
| | 1.00 Mexoryl SX | Terephthalidene Dicamphor Sulfonic |
| | | Acid |
| | 0.20 Keltrol | Xanthan Gum |
| | 0.06 Edeta BD | Disodium EDTA |
| | 0.04 TEA | Triethanolamine |
| | 0.30 Carbopol Ultrez 10P | Carbomer |
| | 63.50 Water dem. | Aqua dem. |
| | 1% MIP | |
| D | q.s. Perfume oil | |
| | 0.50 Glydant | DMDM Hydantoin |

| | | |
|---|---|---|
| A | 5.50 Uvinul MC 80 | Ethylhexyl Methoxycinnamate |
| | 2.00 Uvinul A Plus | Diethylamino Hydroxybenzoyl |
| | | Hexyl Benzoate |
| | 3.00 Uvinul N 539 T | Octocrylene |
| | 3.00 Emulgade PL 68/50 | Cetearyl Glucoside, Cetearyl |
| | | Alcohol |
| | 2.00 Dracorin 100 SE | Glyceryl Stearate, PEG-100 Stearate |
| | 1.00 Fitoderm | Squalane |
| | 0.50 Cremophor WO 7 | PEG-7 Hydrogenated Castor Oil |
| | 0.50 Cremophor PS 20 | Polysorbate 20 |
| | 2.00 Dry Flo Pure | Aluminum Starch Octenylsuccinate |
| B | 5.00 T-Lite SF-S | Titanium Dioxide, Silica Hydrate, |
| | | Alumina Hydrate, Methicone/ |
| | | Dimethicone Copolymer |
| C | 4.00 1,2-Propylene Glycol Care | Propylene Glycol |
| | 0.20 Keltrol | Xanthan Gum |
| | 2.00 Simulgel NS | Hydroxyethyl Acrylate/Sodium |
| | | Acryloyldimethyl Taurate |
| | | Copolymer, Squalane, Polysorbate 60 |
| | 66.30 Water dem. | Aqua dem. |
| | q.s. Preservative | |
| | 0.50 Vitamin E acetate | Tocopheryl Acetate |
| | 0.5% MIP | |
| | 1.00 RetiSTAR | Caprylic/Capric Triglyceride, |
| | | Sodium Ascorbate, Tocopherol, |
| | | Retinol |

| | |
|---|---|
| 5.00 Z-COTE MAX | Zinc Oxide (and) Diphenyl Capryl |
| | Methicone |
| 2.00 Parsol 1789 | Butyl Methoxydibenzoylmethane |
| 1.00 Uvinul T150 | Ethylhexyl Triazone |
| 5.00 Uvinul MC 80 | Ethylhexyl Methoxycinnamate |
| 2.00 Uvinul T 539 N | Octocrylene |
| 0.50 Abil 350 | Dimethicone |
| 2.75 Carnico wax LT 20 | Carnauba (Copernica Cerifera) |
| | Wax, Paraffin |
| 3.70 Candelilla wax LT 281 | Candelilla (Euphorbia Cerifera) |
| LJ | Wax |
| 1.80 Beeswax 3050 PH | Bees Wax |
| 3.20 TeCero wax 30445 | Microcrystalline Wax |
| 3.20 TeCero wax 1030 K | Microcrystalline Wax |
| 1.34 Cutina CP | Cetyl Palmitate |
| 6.40 Vaseline | Petrolatum |
| 7.30 Softisan 100 | Hydrogenated Coco-Glycerides |
| 10.00 Luvitol EHO | Cetearyl Ethylhexanoate |
| 0.17 Bisabolol nat. | Bisabolol |
| 1.84 Vitamin E acetate | Tocopheryl Acetate |
| 0.42 D,L-alpha-Tocopherol | Tocopherol |
| 1% MIP | |
| 40.38 Castor oil | Castor (Ricinus Communis) Oil |

| | |
|---|---|
| 5.00 T-Lite SF | Titanium Dioxide, Alumina |
| | Hydrate, Dimethicone/Methicone |
| | Copolymer |
| 2.00 Parsol 1789 | Butyl Methoxydibenzoylmethane |
| 1.00 Uvinul T150 | Ethylhexyl Triazone |
| 3.00 Uvinul MC 80 | Ethylhexyl Methoxycinnamate |
| 2.00 Uvinul N 539 T | Octocrylene |
| 0.50 Abil 350 | Dimethicone |
| 2.75 Carnico wax LT 20 | Carnauba (Copernica Cerifera) |
| | Wax, Paraffin |
| 3.70 Candelilla wax LT 281 | Candelilla (Euphorbia Cerifera) |
| LJ | Wax |
| 1.80 Beeswax 3050 PH | Bees Wax |
| 3.20 TeCero wax 30445 | Microcrystalline Wax |
| 3.20 TeCero wax 1030 K | Microcrystalline Wax |
| 1.34 Cutina CP | Cetyl Palmitate |
| 6.40 Vaseline | Petrolatum |
| 7.30 Softisan 100 | Hydrogenated Coco-Glycerides |
| 10.00 Luvitol EHO | Cetearyl Ethylhexanoate |
| 0.17 Bisabolol nat. | Bisabolol |
| 1.84 Vitamin E acetate | Tocopheryl Acetate |
| 0.42 D,L-alpha-Tocopherol | Tocopherol |
| 1% MIP | |
| 40.38 Castor oil | Castor (Ricinus Communis) Oil |

| | | |
|---|---|---|
| A | 1.00 Uvinul A Plus | Diethylamino Hydroxybenzoyl |
| | | Hexyl Benzoate |
| | 1.00 Tinosorb S | Bis-Ethylhexyloxyphenol |
| | | Methoxyphenyl Triazine |
| | 3.00 Uvinul MC 80 | Ethylhexyl Methoxycinnamate |
| | 8.00 Miglyol 812 | Caprylic/Capric Triglyceride |
| | 1.50 Dow Corning 350 Fluid | Dimethicone |
| | 3.00 Z-COTE MAX | Titanium Dioxide, Alumina |
| | | Hydrate, Dimethicone/ |
| | | Methicone Copolymer |
| | 3.00 Finsolv TN | C12-15 Alkyl Benzoate |
| | 1.00 Cremophor CO 40 | PEG-40 Hydrogenated Castor |
| | | Oil |
| B | 2.00 Luvigel EM | Caprylic/Capric Triglyceride, |
| | | Sodium Acrylates Copolymer |
| C | 54.80 Water dem, | Aqua dem. |
| D | 15.00 Ethanol 96% | Alcohol |
| | 5.00 1,2-Propylene Glycol Care | Propylene Glycol |
| | 0.50 Cremophor A 25 | Ceteareth-25 |
| | 1.0% MIP | |
| | 1.00 Vitamin E acetate | Tocopheryl Acetate |
| | 0.20 Bisabolol rac. | Bisabolol |

| | | |
|---|---|---|
| A | 25.00 Dow Corning 345 Fluid | Cyclopentasiloxane, |
| | | Cyclohexasiloxane |
| | 20.00 Dow Corning 245 Fluid | Cyclopentasiloxane |
| | 8.00 Uvinul MC 80 | Ethylhexyl |
| | | Methoxycinnamate |
| | 4.00 Abil EM 90 | Cetyl PEG/PPG-10/1 |
| | | Dimethicone |
| | 7.00 T-Lite SF-S | Titanium Dioxide, Silica |
| | | Hydrate, Alumina Hydrate, |
| | | Methicone/Dimethicone |
| | | Copolymer |
| B | 17.00 Ethanol 95% | Alcohol |
| | 9.99 Z-COTE MAX | Zinc Oxide (and) Diphenyl |
| | | Capryl Methicone |
| | 5.00 Water dem. | Aqua dem. |
| | 3.00 Glycerol 87% | Glycerin |
| | 0.01% MIP | |
| | 1.00 Talc | Talc |

| | | |
|---|---|---|
| A | 20.00 Dow Corning 345 Fluid | Cyclopentasiloxane, |
| | | Cyclohexasiloxane |
| | 10.00 Dow Corning 245 Fluid | Cyclopentasiloxane |
| | 7.50 Uvinul MC 80 | Ethylhexyl |
| | | Methoxycinnamate |
| | 2.00 Uvinul T 150 | Ethylhexyl Triazone |
| | 12.00 Cosmacol EMI | Di-C12-13 Alkyl Malate |
| | 4.00 Abil EM 90 | Cetyl PEG/PPG-10/1 |
| | | Dimethicone |
| B | 7.00 T-Lite SF | Titanium Dioxide, Alumina |
| | | Hydrate, Dimethicone/ |
| | | Methicone Copolymer |
| C | 15.00 Ethanol 95% | Alcohol |
| | 10.00 Z-COTE MAX | Zinc Oxide (and) Diphenyl |
| | | Capryl Methicone |
| | 3.00 Glycerol 87% | Glycerin |
| | 1.5% MIP | |
| | 8.00 Water dem. | Aqua dem. |

| | | |
|---|---|---|
| A | 10.00 Uvinul A Plus B | Ethylhexyl Methoxycinnamate, |
| | | Diethylamino Hydroxybenzoyl Hexyl |
| | | Benzoate |
| | 10.00 Uvinul N 539 T | Octocrylene |
| | 4.00 Eumulgin VL 75 | Lauryl Glucoside, Polyglyceryl-2 |
| | | DipolyhydroxyStearate, Glycerin |
| | 8.00 Cetiol B | Dibutyl Adipate |
| | 8.00 Finsolv TN | C12-15 Alkyl Benzoate |
| | 12.00 Myritol 331 | Cocoglycerides |
| | 1.00 Lanette E | Sodium Cetearyl Sulfate |
| | 2.00 Lanette O | Cetearyl Alcohol |
| B | 3.00 Z-COTE MAX | Zinc Oxide (and) Diphenyl Capryl |
| | | Methicone |
| C | 35.08 Water dem, | Aqua dem. |
| | 0.38 Citric acid | Citric Acid |
| | 2.9 Glycerol 87% | Glycerin |
| | 0.05 Edeta BD | Disodium EDTA |
| | 0.20 Allantoin | Allantoin |
| | 0.30 Keltrol | Xanthan Gum |
| | 0.1% MIP | |
| | 1.50 Veegum Ultra | Magnesium Aluminum Silicate |
| D | 0.50 Phenonip | |
| | 1.00 Vitamin E acetate | Tocopheryl Acetate |

1-12. (canceled)

13. A process for the preparation of a cosmetic or dermatological formulation comprising
at least one active compound,
at least one polymer which is molecularly imprinted in the presence of this active compound and
at least one fatty phase,
wherein the polymer comprises, in copolymerized form,
a) at least one compound having a double bond which can be polymerized by means of free-radical polymerization and
b) at least one compound having at least two non-conjugated double bonds which can be polymerized by means of free-radical polymerization,
at least one compound a) being chosen from a1) anionic or anionogenic compounds which can be polymerized by means of free-radical polymerization and
the molar ratio of compound a) to compound b) being in the range of from 1:2 to 1:4, the process comprising preparing the polymer by precipitation polymerization in the presence of the active compound.

14. The process according to claim 13, wherein
1) at least one compound a) is mixed with at least one active compound in a suitable solvent, at least one compound b) is added and the polymerization is started or
2) at least one compound a) is mixed with at least one active compound b) in a suitable solvent and the polymerization is then started.

15. A method for treatment of keratin surfaces, which comprises bringing the keratin surface into contact with a cosmetic or dermatological formulation prepared by the process of claim 13.

16. The process of claim 13, wherein the rate of release of the active compound from the polymer-active compound complex in the cosmetic or dermatological formulation is higher at pH 5 than at pH 7.

17. The process of claim 13, wherein the cosmetic or dermatological formulation comprises at least one compound a) chosen from the group consisting of
a2) esters of α,β-ethylenically unsaturated carboxylic acids
a3) amides of α,β-ethylenically unsaturated carboxylic acids
a4) esters of vinyl alcohol or allyl alcohol with C₁-C₃₀-monocarboxylic acids, vinyl ethers, vinylaromatics, vinyllactams, vinylimidazoles, vinyl halides, vinylidene halides, C₂-C₈-monoolefines, non-aromatic hydrocarbons having at least 2 conjugated double bonds and
a5) mixtures thereof.

18. The process of claim 13, wherein compound a1) in the cosmetic or dermatological formulation is chosen from compounds which can be polymerized by means of free-radical polymerization and contain optionally deprotonated COOH groups.

19. The process of claim 13, wherein the cosmetic or dermatological formulation has a weight ratio of polymer to active compound of 1:10 to 100:1.

20. The process of claim 13, wherein the cosmetic or dermatological formulation is in the form of a cream, foam, spray, gel, gel spray, lotion, oil, oily gel or mousse.
